(19) United States
(12) Patent Application Publication
(10) Pub. No.: US 2026/0097012 A1
(43) Pub. Date: Apr. 9, 2026

(54) COMPOSITIONS AND METHODS FOR TREATING GRAFT-RELATED ARRHYTHMIA

(71) Applicant: BlueRock Therapeutics LP, Cambridge, MA (US)

(72) Inventors: Ibrahim Domian, Swampscott, MA (US); Rocco Romagnuolo, Toronto (CA)

(73) Assignee: BlueRock Therapeutics LP, Cambridge, MA (US)

(21) Appl. No.: 19/115,880

(22) PCT Filed: Sep. 27, 2023
(86) PCT No.: PCT/US2023/075246

§ 371 Date: Mar. 27, 2025

Related U.S. Application Data
(60) Provisional application No. 63/411,087, filed on Sep. 28, 2022.

(51) Int. Cl. A61K 31/343; A61K 31/138; A61K 31/166; A61K 31/167; A61K 31/277; A61K 31/55; A61K 35/34; A61P 9/06

(52) U.S. Cl. CPC A61K 31/343; A61K 31/138; A61K 31/166; A61K 31/167; A61K 31/277; A61K 31/55; A61K 35/34; A61P 9/06

(57) ABSTRACT

Provided herein are methods of treating or preventing an arrhythmia in a cardiac graft patient, wherein the method comprises administering combinations of anti-arrhythmic agents (e.g., amiodarone, metoprolol, ivabradine, and/or mexiletine) to the subject in need thereof.

### RELATED APPLICATIONS

The present application claims priority under 35 U.S.C. § 119(e) to U.S. provisional application, U.S. Ser. No. 63/411,087, filed Sep. 28, 2022, which is incorporated herein by reference.

### BACKGROUND

Pluripotent stem cell-derived cardiomyocytes (PSC-CMs) have been used in small and large animal models for their potential to regenerate injured hearts. PSC-CMs can engraft and survive within the scar region of various animal models and can improve systolic function (e.g., as measured by ejection fraction) or cardiac dimension (e.g., myocardium wall thickness, or ventricular systolic or diastolic internal cavity dimensions) in small animal models compared to vehicle alone. However, in large animal models such as non-human primates and swine, graft-related arrhythmias are observed following transplantation of PSC-CMs. In swine, the graft-related arrhythmias are more frequent and lethal compared to those in small animal models or those with higher baseline heartrate (such as primates with higher heart rates). Significantly reducing or eliminating graft-related arrhythmias remains a top priority to using PSC-CMs as a viable therapy to treat patients with heart disease.

### SUMMARY

Cardiac grafts (e.g., transplantation of cardiomyocytes) are a treatment of heart failure in subjects but may induce arrhythmia. Provided herein are methods of treating arrhythmia (e.g., graft-induced arrhythmia) in a subject who is in need of or has received a cardiac graft, wherein the method comprises administering one or more anti-arrhythmic agents (e.g., anti-arrhythmic compounds). The present disclosure also provides pharmaceutical compositions and kits. The provided methods and kits may be useful in reducing arrhythmia induced by cardiac grafts.

In some embodiments, at least one of the anti-arrhythmic agents is a Class III anti-arrhythmic agent (e.g., amiodarone, or an analog or derivative thereof, or a pharmaceutically acceptable salt, isotopically labeled compound, tautomer, stereoisomer, solvate, tautomer, hydrate, polymorph, co-crystal, or prodrug thereof).

In some embodiments, at least one of the anti-arrhythmic agents is a Class II anti-arrhythmic agent (e.g., metoprolol, or an analog or derivative thereof, or a pharmaceutically acceptable salt, isotopically labeled compound, tautomer, stereoisomer, solvate, tautomer, hydrate, polymorph, co-crystal, or prodrug thereof). In some embodiments, at least one of the anti-arrhythmic agents is a beta-blocker.

In some embodiments, at least one of the anti-arrhythmic agents is a Class 0 anti-arrhythmic agent (e.g., ivabradine, or an analog or derivative thereof, or a pharmaceutically acceptable salt, isotopically labeled compound, tautomer, stereoisomer, solvate, tautomer, hydrate, polymorph, co-crystal, or prodrug thereof).

In some embodiments, at least one of the anti-arrhythmic agents is a Class I (e.g., Class Ib) anti-arrhythmic agent (e.g., mexiletine or lidocaine; or an analog or derivative thereof, or a pharmaceutically acceptable salt, isotopically labeled compound, tautomer, stereoisomer, solvate, tautomer, hydrate, polymorph, co-crystal, or prodrug thereof).

In some embodiments, at least one of the anti-arrhythmic agents is a Class IV anti-arrhythmic agent (e.g., verapamil, or an analog or derivative thereof, or a pharmaceutically acceptable salt, isotopically labeled compound, tautomer, stereoisomer, solvate, tautomer, hydrate, polymorph, co-crystal, or prodrug thereof).

In some embodiments, at least one of the anti-arrhythmic agents is a Class I (e.g., Class Ia) anti-arrhythmic agent (e.g., procainamide, or an analog or derivative thereof, or a pharmaceutically acceptable salt, isotopically labeled compound, tautomer, stereoisomer, solvate, tautomer, hydrate, polymorph, co-crystal, or prodrug thereof).

In some embodiments, the method comprises administering two or more anti-arrhythmic agents. In some embodiments, the method comprises administering two or more of amiodarone, metoprolol, and ivabradine, optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof. In some aspects, the method further comprises administering mexiletine, or an analog or derivative thereof, or a pharmaceutically acceptable salt, isotopically labeled compound, tautomer, stereoisomer, solvate, tautomer, hydrate, polymorph, co-crystal, or prodrug thereof, to the subject in need thereof. In other aspects, the method further comprises administering lidocaine, or an analog or derivative thereof, or a pharmaceutically acceptable salt, isotopically labeled compound, tautomer, stereoisomer, solvate, tautomer, hydrate, polymorph, co-crystal, or prodrug thereof, to the subject in need thereof. In other aspects, the method further comprises administering procainamide, or an analog or derivative thereof, or a pharmaceutically acceptable salt, isotopically labeled compound, tautomer, stereoisomer, solvate, tautomer, hydrate, polymorph, co-crystal, or prodrug thereof, to the subject in need thereof. In other aspects, the method further comprises administering verapamil, or an analog or derivative thereof, or a pharmaceutically acceptable salt, isotopically labeled compound, tautomer, stereoisomer, solvate, tautomer, hydrate, polymorph, co-crystal, or prodrug thereof, to the subject in need thereof.

In some aspects, provided herein are methods of treating a cardiovascular disease, disorder, or symptom thereof, wherein the method comprises administering cardiomyocytes to a subject in need thereof. In some aspects, the cardiomyocytes are administered prior to, concurrently with, or subsequent to the administration of any one or two or three of amiodarone, metoprolol, and ivabradine, optionally each of which independently comprises an analog or derivative thereof, or a pharmaceutically acceptable salt, isotopically labeled derivative, or prodrug thereof.

In another aspect, the present disclosure provides kits comprising: (1) two or more of amiodarone, metoprolol, ivabradine, and mexiletine, optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof; and (2) instructions for administering (1) to treat or prevent arrhythmia in a subject in need thereof.

The details of one or more embodiments of the present disclosure are set forth herein. Other features, objects, and advantages of the present disclosure will be apparent from the Detailed Description, Examples, Figures, and Claims.

### BRIEF DESCRIPTION OF THE DRAWINGS

FIG. 1 shows percentage of day a test pig remains in sustained VT for test pigs subjected to an infarct, a cardiac graft of immature cells, and administered the specified combination of anti-arrhythmic agents. Test pigs underwent thoracotomy and received the cardiac graft at day 0.

FIG. 2 shows ECG traces for various pigs subjected to an infarct, a cardiac graft of either mature (P6) or immature (P2, P4, and P5) cardiomyocytes.

### DETAILED DESCRIPTION

This disclosure is based, in part, on the surprising finding that subjects treated with two or more of amiodarone, metoprolol, and ivabradine prior to, concurrently with, or subsequent to a cardiac graft experience reduced graft-induced arrhythmias.

### Methods of Use

Provided herein is a method comprising administering two or more of: amiodarone, metoprolol, and ivabradine, optionally each of which independently comprises an analog or derivative thereof, or a pharmaceutically acceptable salt, solvate, tautomer, hydrate, polymorph, co-crystal, isotopically labeled compound, or prodrug thereof; to a subject in need thereof, wherein the subject is in need of or has received a cardiac graft.

Amiodarone is a class III anti-arrhythmic agent and is indicated for the treatment of, e.g., cardiac arrest, ventricular tachycardia, and atrial fibrillation. Amiodarone may inhibit voltage-gated potassium channels and voltage-gated calcium channels, which in turn may prolong phase 3 of the cardiac action potential. Amiodarone may inhibit the pore-forming subunit of the potassium ion channel, KV11.1 (encoded by the KCNH2 gene). Amiodarone may inhibit the voltage-gated calcium channel (encoded by the CACNA2D2 gene). Amiodarone may inhibit voltage-gated sodium channel activity. Analogs and derivatives of amiodarone are known in the art, see e.g., U.S. Pat. Nos. 7,799,799; 9,018,250; and Carlsson et al., J. Med. Chem. 2002, 45, 623-30, each of which is incorporated herein by reference in its entirety. Amiodarone analogs and derivatives may also be useful in the methods provided herein. In some embodiments, the amiodarone analog or derivative is dronedarone. In some embodiments, amiodarone, or an analog or derivative thereof, or a pharmaceutically acceptable salt, solvate, tautomer, hydrate, polymorph, co-crystal, isotopically labeled compound, or prodrug thereof, is amiodarone, or a pharmaceutically acceptable salt thereof. In some embodiments, amiodarone, or an analog or derivative thereof, or a pharmaceutically acceptable salt, solvate, tautomer, hydrate, polymorph, co-crystal, isotopically labeled compound, or prodrug thereof, is in a long-acting or short-acting, and/or fast-acting or slow-acting, and/or fast-release or extended-release form. In some embodiments, amiodarone, or an analog or derivative thereof, or a pharmaceutically acceptable salt, solvate, tautomer, hydrate, polymorph, co-crystal, isotopically labeled compound, or prodrug thereof, is in a form suitable for oral or intravenous administration.

Metoprolol is a class II anti-arrhythmic agent and is indicated for the treatment of, e.g., high blood pressure, chest pain due to poor blood flow to the heart, and a number of conditions involving an abnormally fast heart rate. Metoprolol is a selective β1 receptor blocker. By working on the β1 receptor of the cardiac muscle cells, metoprolol may yield both a chronotropic and inotropic effect. Metoprolol analogs and derivatives may also be useful in the methods provided herein. In some embodiments, the metoprolol analog or derivative is esmolol. In some embodiments, metoprolol, or an analog or derivative thereof, or a pharmaceutically acceptable salt, solvate, tautomer, hydrate, polymorph, co-crystal, isotopically labeled compound, or prodrug thereof, is metoprolol, or a pharmaceutically acceptable salt thereof. In some embodiments, metoprolol, or an analog or derivative thereof, or a pharmaceutically acceptable salt, solvate, tautomer, hydrate, polymorph, co-crystal, isotopically labeled compound, or prodrug thereof, is in a long-acting or short-acting, and/or fast-acting or slow-acting, and/or fast-release or extended-release form. In some embodiments, metoprolol, or an analog or derivative thereof, or a pharmaceutically acceptable salt, solvate, tautomer, hydrate, polymorph, co-crystal, isotopically labeled compound, or prodrug thereof, is in a form suitable for oral or intravenous administration.

In some embodiments, at least one of the anti-arrhythmic agents is a non-selective beta-blocker. In some embodiments, at least one of the anti-arrhythmic agents is a selective beta-blocker. In some embodiments, the selective beta-blocker blocks one or more beta (e.g., beta-1, beta-2, or beta-1 and 2) receptors more than other beta (e.g., beta-3) receptors. In some embodiments, the selective beta-blocker is cardioselective (e.g., selective for beta-1 receptors). In some embodiments, the beta-1 selective beta blocker is acebutolol, atenolol, betaxolol, bisoprolol, esmolol, metoprolol, or nebivolol, or an analog or derivative thereof, or a pharmaceutically acceptable salt, isotopically labeled compound, tautomer, stereoisomer, solvate, tautomer, hydrate, polymorph, co-crystal, or prodrug thereof. In some embodiments, the non-selective beta blocker is carvedilol, labetalol, nadolol, penbutolol, pindolol, propranolol, sotalol, or timolol, or an analog or derivative thereof, or a pharmaceutically acceptable salt, isotopically labeled compound, tautomer, stereoisomer, solvate, tautomer, hydrate, polymorph, co-crystal, or prodrug thereof.

Ivabradine is a class 0 anti-arrhythmic agent and is indicated for the treatment of, e.g., heart-related chest pain and heart failure. Ivabradine is a funny current (If) inhibitor. Ivabradine may allow negative chronotropy in the sinoatrial structure thus reducing the heart rate via specific inhibition of the pacemaker current, a mechanism different from that of beta blockers and calcium channel blockers. Ivabradine analogs and derivatives may also be useful in the methods provided herein. In some embodiments, ivabradine, or an analog or derivative thereof, or a pharmaceutically acceptable salt, solvate, tautomer, hydrate, polymorph, co-crystal, isotopically labeled compound, or prodrug thereof, is ivabradine, or a pharmaceutically acceptable salt thereof. In some embodiments, ivabradine, or an analog or derivative thereof, or a pharmaceutically acceptable salt, solvate, tautomer, hydrate, polymorph, co-crystal, isotopically labeled compound, or prodrug thereof, is in a long-acting or short-acting, and/or fast-acting or slow-acting, and/or fast-release or extended-release form. In some embodiments, ivabradine, or an analog or derivative thereof, or a pharmaceutically acceptable salt, solvate, tautomer, hydrate, polymorph, co-crystal, isotopically labeled compound, or prodrug thereof, is in a form suitable for oral or intravenous administration.

In some embodiments, the method comprises administering amiodarone, metoprolol, and ivabradine, optionally each of which independently comprises an analog or derivative thereof, or a pharmaceutically acceptable salt, isotopically labeled derivative, or prodrug thereof; to a subject in need thereof, wherein the subject is in need of or has received a cardiac graft. In some embodiments, the method comprises administering amiodarone and ivabradine, optionally each of which independently comprises an analog or derivative thereof, or a pharmaceutically acceptable salt thereof; to a subject in need thereof, wherein the subject is in need of or has received a cardiac graft.

In some embodiments, the method further comprises administering mexiletine, or an analog or derivative thereof, or a pharmaceutically acceptable salt, solvate, tautomer, hydrate, polymorph, co-crystal, isotopically labeled compound, or prodrug thereof, to the subject in need thereof. In some embodiments, the method further comprises administering mexiletine, or a pharmaceutically acceptable salt thereof, to the subject in need thereof. In some embodiments, the method further comprises administering mexiletine to the subject in need thereof.

Mexiletine is a class Ib anti-arrhythmic agent and is indicated for the treatment of, e.g., abnormal heart rhythms, chronic pain, and some causes of muscle stiffness. Mexiletine is a non-selective voltage-gated sodium channel blocker. Mexiletine analogs and derivatives may also be useful in the methods provided herein. In some embodiments, mexiletine, or an analog or derivative thereof, or a pharmaceutically acceptable salt, solvate, tautomer, hydrate, polymorph, co-crystal, isotopically labeled compound, or prodrug thereof, is mexiletine, or a pharmaceutically acceptable salt thereof. In some embodiments, mexiletine, or an analog or derivative thereof, or a pharmaceutically acceptable salt, solvate, tautomer, hydrate, polymorph, co-crystal, isotopically labeled compound, or prodrug thereof, is in a long-acting or short-acting, and/or fast-acting or slow-acting, and/or fast-release or extended-release form. In some embodiments, mexiletine, or an analog or derivative thereof, or a pharmaceutically acceptable salt, solvate, tautomer, hydrate, polymorph, co-crystal, isotopically labeled compound, or prodrug thereof, is in a form suitable for oral or intravenous administration.

In some embodiments, the method comprises administering amiodarone and metoprolol, optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof, to the subject in need thereof. In some embodiments, the method comprises administering amiodarone and ivabradine, optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof, to the subject in need thereof. In some embodiments, the method does not comprise administering amiodarone, or an analog or derivative thereof, or a pharmaceutically acceptable salt, isotopically labeled compound, tautomer, stereoisomer, solvate, tautomer, hydrate, polymorph, co-crystal, or prodrug thereof, to the subject in need thereof. In some embodiments, the method does not comprise administering ivabradine, or an analog or derivative thereof, or a pharmaceutically acceptable salt, isotopically labeled compound, tautomer, stereoisomer, solvate, tautomer, hydrate, polymorph, co-crystal, or prodrug thereof, to the subject in need thereof. In some embodiments, the method comprises administering metoprolol and ivabradine, optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof, to the subject in need thereof. In some embodiments, the method comprises administering amiodarone, metoprolol, and ivabradine, optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof, to the subject in need thereof. In some embodiments, the method comprises administering amiodarone, metoprolol, and mexiletine, optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof, to the subject in need thereof. In some embodiments, the method comprises administering amiodarone, ivabradine, and mexiletine, optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof, to the subject in need thereof. In some embodiments, the method comprises administering metoprolol, ivabradine, and mexiletine, optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof, to the subject in need thereof. In some embodiments, the method comprises administering amiodarone, metoprolol, ivabradine, and mexiletine, optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof, to the subject in need thereof.

In some embodiments, the method further comprises administering lidocaine, or an analog or derivative thereof, or a pharmaceutically acceptable salt, solvate, tautomer, hydrate, polymorph, co-crystal, isotopically labeled compound, or prodrug thereof to the subject in need thereof. In some embodiments, the method further comprises administering lidocaine, or a pharmaceutically acceptable salt thereof, to the subject in need thereof. In some embodiments, the method further comprises administering lidocaine to the subject in need thereof.

Lidocaine is a class Ib anti-arrhythmic agent and is indicated for the treatment of, e.g., pain and ventricular tachycardia. Lidocaine is a sodium channel blocker. Lidocaine analogs and derivatives may also be useful in the methods provided herein. In some embodiments, lidocaine, or an analog or derivative thereof, or a pharmaceutically acceptable salt, solvate, tautomer, hydrate, polymorph, co-crystal, isotopically labeled compound, or prodrug thereof, is lidocaine, or a pharmaceutically acceptable salt thereof. In some embodiments, lidocaine, or an analog or derivative thereof, or a pharmaceutically acceptable salt, solvate, tautomer, hydrate, polymorph, co-crystal, isotopically labeled compound, or prodrug thereof, is in a long-acting or short-acting, and/or fast-acting or slow-acting, and/or fast-release or extended-release form. In some embodiments, lidocaine, or an analog or derivative thereof, or a pharmaceutically acceptable salt, solvate, tautomer, hydrate, polymorph, co-crystal, isotopically labeled compound, or prodrug thereof, is in a form suitable for oral or intravenous administration.

In some embodiments, the method further comprises administering verapamil, or an analog or derivative thereof, or a pharmaceutically acceptable salt, solvate, tautomer, hydrate, polymorph, co-crystal, isotopically labeled compound, or prodrug thereof to the subject in need thereof. In some embodiments, the method further comprises administering verapamil, or a pharmaceutically acceptable salt thereof, to the subject in need thereof. In some embodiments, the method further comprises administering verapamil to the subject in need thereof.

Verapamil is a class IV anti-arrhythmic agent and is indicated for the treatment of, e.g., high blood pressure, angina, and supraventricular tachycardia. Verapamil is a calcium channel blocker. Verapamil analogs and derivatives may also be useful in the methods provided herein. In some embodiments, verapamil, or an analog or derivative thereof, or a pharmaceutically acceptable salt, solvate, tautomer, hydrate, polymorph, co-crystal, isotopically labeled compound, or prodrug thereof, is verapamil, or a pharmaceutically acceptable salt thereof. In some embodiments, verapamil, or an analog or derivative thereof, or a pharmaceutically acceptable salt, solvate, tautomer, hydrate, polymorph, co-crystal, isotopically labeled compound, or prodrug thereof, is in a long-acting or short-acting, and/or fast-acting or slow-acting, and/or fast-release or extended-release form. In some embodiments, verapamil, or an analog or derivative thereof, or a pharmaceutically acceptable salt, solvate, tautomer, hydrate, polymorph, co-crystal, isotopically labeled compound, or prodrug thereof, is in a form suitable for oral or intravenous administration.

In some embodiments, the method further comprises administering procainamide, or an analog or derivative thereof, or a pharmaceutically acceptable salt, solvate, tautomer, hydrate, polymorph, co-crystal, isotopically labeled compound, or prodrug thereof to the subject in need thereof. In some embodiments, the method further comprises administering procainamide, or a pharmaceutically acceptable salt thereof, to the subject in need thereof. In some embodiments, the method further comprises administering procainamide to the subject in need thereof.

Procainamide is a class Ia anti-arrhythmic agent and is indicated for the treatment of, e.g., cardiac arrhythmias. Procainamide is a sodium channel blocker of cardiomyocytes. In addition to blocking the INa current, procainamide may inhibit the IKr rectifier K+ current. Procainamide may also induce a voltage-dependent open channel block on the batrachotoxin (BTX)-activated sodium channels in cardiomyocytes. Procainamide analogs and derivatives may also be useful in the methods provided herein. In some embodiments, procainamide, or an analog or derivative thereof, or a pharmaceutically acceptable salt, solvate, tautomer, hydrate, polymorph, co-crystal, isotopically labeled compound, or prodrug thereof, is procainamide, or a pharmaceutically acceptable salt thereof. In some embodiments, procainamide, or an analog or derivative thereof, or a pharmaceutically acceptable salt, solvate, tautomer, hydrate, polymorph, co-crystal, isotopically labeled compound, or prodrug thereof, is in a long-acting or short-acting, and/or fast-acting or slow-acting, and/or fast-release or extended-release form. In some embodiments, procainamide, or an analog or derivative thereof, or a pharmaceutically acceptable salt, solvate, tautomer, hydrate, polymorph, co-crystal, isotopically labeled compound, or prodrug thereof, is in a form suitable for oral or intravenous administration.

In some embodiments, the method further comprises administering lidocaine and procainamide, optionally each of which independently comprises an analog or derivative thereof, or a pharmaceutically acceptable salt, solvate, tautomer, hydrate, polymorph, co-crystal, isotopically labeled compound, or prodrug thereof to the subject in need thereof. In some embodiments, the method further comprises administering lidocaine and procainamide, or a pharmaceutically acceptable salt thereof, to the subject in need thereof. In some embodiments, the method further comprises administering lidocaine and procainamide to the subject in need thereof.

In some embodiments, the method comprises administering two or more of amiodarone, metoprolol, ivabradine, and mexiletine, optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof, to the subject in need thereof. In some embodiments, the method comprises administering three or more of amiodarone, metoprolol, ivabradine, and mexiletine, optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof, to the subject in need thereof. In some embodiments, the method comprises administering amiodarone, metoprolol, ivabradine, and mexiletine, optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof, to the subject in need thereof.

In some embodiments, the subject is at risk of or suffers from a cardiovascular disease. In some embodiments, the cardiovascular disease is a cardiomyopathy. In some embodiments, the cardiomyopathy is heart failure. In some embodiments, the cardiomyopathy is myocardial infarction. In certain embodiments, the cardiovascular disease is ischemic cardiomyopathy, dilated cardiomyopathy, or heart failure associated with prior myocardial infarction. In certain embodiments, the subject suffers from a cardiac birth defect or congenital heart disease, optionally hypoplastic left heart syndrome. In some embodiments the subject may already be on metoprolol, or an analog or derivative thereof, or a pharmaceutically acceptable salt, isotopically labeled compound, tautomer, stereoisomer, solvate, tautomer, hydrate, polymorph, co-crystal, or prodrug thereof, or other beta blocker therapy. In some embodiments, the beta blocker may be clinically indicated independent of cardiomyocyte grafts.

In some embodiments, subject is at risk of or suffers from arrhythmia (e.g., a graft-induced arrhythmia). A graft-induced arrhythmia may be caused the presence of an arrhythmogenic cell population in a graft. In some embodiments, an arrhythmogenic cell population comprises a population of cells for which a cell therapy is enriched, e.g., ventricular cardiomyocytes in a graft applied in a subject's ventricle. In some embodiments, an arrhythmogenic cell population in a graft is a population of cells that is a by-product or contamination in a cell product (e.g., atrial cells, pacemaker cells, or non-cardiac cells in a graft applied in a subject's ventricle). An arrhythmia may be a supraventricular arrhythmia (e.g., paroxysmal supraventricular tachycardia (PSVT), accessory pathway tachycardia, AV nodal re-entrant tachycardia (AVNRT), Atrial tachycardia, Atrial fibrillation, Atrial flutter), a ventricular arrhythmia (e.g., Premature ventricular contractions (PVCs), ventricular tachycardia (V-tach), ventricular fibrillation (V-fib), or long QT), or a bradyarrhythmia (e.g., Sinus node dysfunction or heart block). In some embodiments, an arrhythmia is tachycardia. In some embodiments, an arrhythmia is ventricular tachycardia. In some embodiments, an arrhythmia is caused by a cardiac graft or at least in part by a cardiac graft. In some embodiments, an arrhythmia occurs after the cardiac graft. In some embodiments an arrhythmia is transient. In some embodiments, an arrhythmia is persistent or chronic. In some embodiments, the arrhythmia is caused at least in part by a subpopulation of cells (e.g., atrial cells, pacemaker cells, conduction system cells, and/or other cardiac or non-cardiac subpopulation of cells responsible or contributing to graft associated arrhythmia). In some embodiments, the arrhythmia is caused at least in part by cells determined by CD200 as a marker. In some embodiments, a subject to which any of the disclosed anti-arrhythmic agents are administered, e.g., concurrently/subsequently with cardiomyocyte graft, is using a mechanical pump to assist the heart in pumping blood. Non-limiting examples of a mechanical pump include a left ventricular assist device (LVAD), or an Impella device.

A cardiac graft of the present disclosure may comprise fibroblasts, one or more types of cardiomyocytes (e.g., ventricular cardiomyocytes, atrial cardiomyocytes, and/or smooth muscle cells), endothelial cells, conduction cells and/or pacemaker cells. In some embodiments, the cardiac graft comprises cardiomyocytes. In some embodiments, the cardiomyocytes are ventricular cardiomyocytes. In some embodiments, the cardiomyocytes are immature cardiomyocytes. In some embodiments, the cardiomyocytes are immature ventricular cardiomyocytes. In some embodiments, the cardiomyocytes are mature cardiomyocytes. In some embodiments, the cardiomyocytes are mature ventricular cardiomyocytes. In some embodiments, the cardiomyocytes are made from pluripotent stem cells (e.g., induced pluripotent stem cells). In some embodiments, cardiomyocytes may be immune evading or hypoimmune. In certain embodiments, the cardiac graft further comprises fibroblasts, other types of cardiomyocytes (e.g., atrial cardiomyocytes or smooth muscle cells), endothelial cells, conduction cells and/or pacemaker cells.

In some embodiments, a mature cardiomyocyte is made by culturing a precursor (e.g., an immature cardiomyocyte) on a substrate (e.g., adherent culture) for at least 2 weeks (e.g., between 2 and 3, between 3 and 4, between 4 and 5, between 5 and 6, between 6 and 8, between 8 and 10, or between 10 and 12 weeks, inclusive, or more than 12 weeks) in culture media that promotes oxidative phosphorylation. In some embodiments, a mature cardiomyocyte is made by a method described in Guo et al., Circulation Research, 2020, 126, 1086-1106, incorporated herein by reference in its entirety.

In certain embodiments, the method further comprises using or implanting a mechanical circulatory support on or to the subject.

In certain embodiments, the method comprises:
administering amiodarone, or an analog or derivative thereof, or a pharmaceutically acceptable salt, isotopically labeled compound, tautomer, stereoisomer, solvate, tautomer, hydrate, polymorph, co-crystal, or prodrug thereof, to the subject in need thereof; and using or implanting a mechanical circulatory support on or to the subject in need thereof.

In some embodiments, a method comprises:
administering ivabradine, or an analog or derivative thereof, or a pharmaceutically acceptable salt, isotopically labeled compound, tautomer, stereoisomer, solvate, tautomer, hydrate, polymorph, co-crystal, or prodrug thereof, to the subject in need thereof; and
using or implanting the mechanical circulatory support on or to the subject in need thereof. In some embodiments, a method as disclosed herein comprises administering to a subject amiodarone, or an analog or derivative thereof, or a pharmaceutically acceptable salt, isotopically labeled compound, tautomer, stereoisomer, solvate, tautomer, hydrate, polymorph, co-crystal, or prodrug thereof, to the subject in need thereof; ivabradine, or an analog or derivative thereof, or a pharmaceutically acceptable salt, isotopically labeled compound, tautomer, stereoisomer, solvate, tautomer, hydrate, polymorph, co-crystal, or prodrug thereof, to the subject in need thereof; and using or implanting the mechanical circulatory support on or to the subject.

In some embodiments a subject may have or may receive mechanical circulatory support before, after, or at the time of cardiomyocyte grafts. In some embodiments the mechanical circulatory support may be a Left Ventricular Assist Device (LVAD), a Right Ventricular Assist Device (RVAD), a Bi Ventricular Assist Device (BiVAD), Extra Corporeal Membrane Oxygenation (ECMO), or Implantable Cardiac Defibrillator (ICD), or a combination thereof. In certain embodiments, the ICD is linked by a biventricular pacer. In some embodiments, mechanical circulatory support may be an intravascular, microaxial blood pump (such as Impella 5.0). In some embodiments, this mechanical circulatory support is ceased after the subject receives a cardiac graft. In some embodiments, the mechanical circulatory support is used or implanted on or to the subject prior to the administration of the cardiac graft.

In some embodiments, grafts may be delivered by open surgical approach, minimally invasive approach, or percutaneous/intravascular approach.

In some embodiments, the one or more of amiodarone, metoprolol, ivabradine, and/or mexiletine, and optionally lidocaine, verapamil, and/or procainamide; optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof, are administered concurrently with administration of the cardiac graft. In some embodiments, the one or more of amiodarone, metoprolol, ivabradine, and/or mexiletine, and optionally lidocaine, verapamil, or procainamide; optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof, are administered subsequent to or prior to administration of the cardiac graft.

In some embodiments, one or more of the amiodarone, metoprolol, ivabradine, and/or mexiletine, and optionally lidocaine, verapamil, or procainamide; optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof, are administered within between 2 and 4 months, between 1 and 2 months, between 4 and 6 weeks, between 2 and 4 weeks, between 1 and 2 weeks, about 7 days, or about 6 days, or about 5 days, or about 4 days, or about 3 days, or about 2 days, or about 1 days, or about 24 hours, or about 20 hours, or about 18 hours, or about 16 hours, or about 14 hours, or about 12 hours, or about 10 hours, or about 8 hours, or about 6 hours, or about 5 hours, or about 4 hours, or about 3 hours, or about 2 hours, or about 1 hour, or about 50 minutes, or about 40 minutes, or about 30 minutes, or about 20 minutes, or about 10 minutes, or about 5 minutes, or about 4 minutes, or about 3 minutes, or about 2 minutes, or about 1 minute of administration of the cardiac graft.

In some embodiments, one or more of the amiodarone, metoprolol, ivabradine, and/or mexiletine, and optionally lidocaine, verapamil, or procainamide; optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof, are administered within about 1-10 weeks, about 1-6 weeks, about 2-6 weeks, about 3-6 weeks, about 2-5 weeks, about 3-4 weeks, about 1 week, about 2 weeks, about 3 weeks, about 4 weeks, about 5 weeks, about 6 weeks, about 7 weeks, or about 8 weeks before the cardiac graft.

In some embodiments, the method described herein comprises administration of one or more of amiodarone, metoprolol, ivabradine, mexiletine, lidocaine, verapamil, and/or procainamide; optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof, prior to administration of the cardiac graft, followed by administration of two or more of amiodarone, metoprolol, ivabradine, mexiletine, lidocaine, verapamil, and/or procainamide; optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof, which can occur prior to, concurrently with, or subsequently to the cardiac graft. In some embodiments, a subject in need thereof may be administered one of the pharmaceutical agents described herein for about 1-6 weeks prior to the cardiac graft, followed by administration 2 or 3 or more of the pharmaceutical agents for about 0-7 days prior to, or concurrently with, the cardiac graft. In some embodiments, a subject in need thereof may be administered one of the pharmaceutical agents described herein for about 1-6 weeks prior to the cardiac graft, followed by administration 2 or 3 or more of the of the pharmaceutical agents described here subsequent to the cardiac graft.

In some embodiments, the administration of amiodarone, or an analog or derivative thereof, or a pharmaceutically acceptable salt, isotopically labeled compound, tautomer, stereoisomer, solvate, tautomer, hydrate, polymorph, co-crystal, or prodrug thereof, is initiated prior to administration of the graft of cardiomyocytes. In some embodiments, administration of amiodarone, or an analog or derivative thereof, or a pharmaceutically acceptable salt, isotopically labeled compound, tautomer, stereoisomer, solvate, tautomer, hydrate, polymorph, co-crystal, or prodrug thereof, can be, for example 1 day before, 2 days before, 3 days before, 4 days before, 5 days before, 6 days before, 1 week before, 2 weeks before, 3 weeks before, 4 weeks before, 5 weeks before, 6 weeks before, or 7 weeks or more prior to the graft.

In some embodiments, the administration of metoprolol, or an analog or derivative thereof, or a pharmaceutically acceptable salt, isotopically labeled compound, tautomer, stereoisomer, solvate, tautomer, hydrate, polymorph, co-crystal, or prodrug thereof, is initiated prior to administration of the graft of cardiomyocytes. In some embodiments, administration of metoprolol, or an analog or derivative thereof, or a pharmaceutically acceptable salt, isotopically labeled compound, tautomer, stereoisomer, solvate, tautomer, hydrate, polymorph, co-crystal, or prodrug thereof, can be, for example 1 day before, 2 days before, 3 days before, 4 days before, 5 days before, 6 days before, 1 week before, 2 weeks before, 3 weeks before, 4 weeks before, 5 weeks before, 6 weeks before, or 7 weeks or more prior to the graft.

In some embodiments, the administration of ivabradine, or an analog or derivative thereof, or a pharmaceutically acceptable salt, isotopically labeled compound, tautomer, stereoisomer, solvate, tautomer, hydrate, polymorph, co-crystal, or prodrug thereof, is initiated prior to administration of the graft of cardiomyocytes. In some embodiments, administration of ivabradine, or an analog or derivative thereof, or a pharmaceutically acceptable salt, isotopically labeled compound, tautomer, stereoisomer, solvate, tautomer, hydrate, polymorph, co-crystal, or prodrug thereof, can be, for example 1 day before, 2 days before, 3 days before, 4 days before, 5 days before, 6 days before, 1 week before, 2 weeks before, 3 weeks before, 4 weeks before, 5 weeks before, 6 weeks before, or 7 weeks or more prior to the graft.

In some embodiments, the administration of mexiletine, or an analog or derivative thereof, or a pharmaceutically acceptable salt, isotopically labeled compound, tautomer, stereoisomer, solvate, tautomer, hydrate, polymorph, co-crystal, or prodrug thereof, is initiated prior to administration of the graft of cardiomyocytes. In some embodiments, administration of mexiletine, or an analog or derivative thereof, or a pharmaceutically acceptable salt, isotopically labeled compound, tautomer, stereoisomer, solvate, tautomer, hydrate, polymorph, co-crystal, or prodrug thereof, can be, for example 1 day before, 2 days before, 3 days before, 4 days before, 5 days before, 6 days before, 1 week before, 2 weeks before, 3 weeks before, 4 weeks before, 5 weeks before, 6 weeks before, or 7 weeks or more prior to the graft.

In some embodiments, the administration of lidocaine, or an analog or derivative thereof, or a pharmaceutically acceptable salt, isotopically labeled compound, tautomer, stereoisomer, solvate, tautomer, hydrate, polymorph, co-crystal, or prodrug thereof, is initiated prior to administration of the graft of cardiomyocytes. In some embodiments, administration of lidocaine, or an analog or derivative thereof, or a pharmaceutically acceptable salt, isotopically labeled compound, tautomer, stereoisomer, solvate, tautomer, hydrate, polymorph, co-crystal, or prodrug thereof, can be, for example 1 day before, 2 days before, 3 days before, 4 days before, 5 days before, 6 days before, 1 week before, 2 weeks before, 3 weeks before, 4 weeks before, 5 weeks before, 6 weeks before, or 7 weeks or more prior to the graft.

In some embodiments, the administration of verapamil, or an analog or derivative thereof, or a pharmaceutically acceptable salt, isotopically labeled compound, tautomer, stereoisomer, solvate, tautomer, hydrate, polymorph, co-crystal, or prodrug thereof, is initiated prior to administration of the graft of cardiomyocytes. In some embodiments, administration of verapamil, or an analog or derivative thereof, or a pharmaceutically acceptable salt, isotopically labeled compound, tautomer, stereoisomer, solvate, tautomer, hydrate, polymorph, co-crystal, or prodrug thereof, can be, for example 1 day before, 2 days before, 3 days before, 4 days before, 5 days before, 6 days before, 1 week before, 2 weeks before, 3 weeks before, 4 weeks before, 5 weeks before, 6 weeks before, or 7 weeks or more prior to the graft.

In some embodiments, the administration of procainamide, or an analog or derivative thereof, or a pharmaceutically acceptable salt, isotopically labeled compound, tautomer, stereoisomer, solvate, tautomer, hydrate, polymorph, co-crystal, or prodrug thereof, is initiated prior to administration of the graft of cardiomyocytes. In some embodiments, administration of procainamide, or an analog or derivative thereof, or a pharmaceutically acceptable salt, isotopically labeled compound, tautomer, stereoisomer, solvate, tautomer, hydrate, polymorph, co-crystal, or prodrug thereof, can be, for example 1 day before, 2 days before, 3 days before, 4 days before, 5 days before, 6 days before, 1 week before, 2 weeks before, 3 weeks before, 4 weeks before, 5 weeks before, 6 weeks before, or 7 weeks or more prior to the graft.

In another embodiment of this or any other aspect, administration of both amiodarone and metoprolol; optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof, is initiated prior to (e.g., 1 day before, 2 days before, 3 days before, 4 days before, 5 days before, 6 days before, or 7 days or more prior to) administration of the cardiac graft of cardiomyocytes.

In some embodiments, any one of the pharmaceutical agents described herein is administered to a subject more than once (e.g., 1, 2, 3, 4, 5, 6, 7, 8, 9, 10 or more times) over a period of hours, days, or even weeks (e.g., between 1 and 3 hours, between 3 and 6 hours, between 6 and 12 hours, between 12 and 24 hours, between 1 and 2 days, between 2 and 4 days, between 4 and 7 days, between 1 and 2 weeks, between 2 and 4 weeks, or between 1 and 2 months, inclusive). In some embodiments, the method further comprises an additional administration of mexiletine, or an analog or derivative thereof, or a pharmaceutically acceptable salt, isotopically labeled compound, tautomer, stereoisomer, solvate, tautomer, hydrate, polymorph, co-crystal, or prodrug thereof, in the same or a different dose conducted 24 or more hours subsequent to a first administration of mexiletine, or an analog or derivative thereof, or a pharmaceutically acceptable salt, isotopically labeled compound, tautomer, stereoisomer, solvate, tautomer, hydrate, polymorph, co-crystal, or prodrug thereof.

“About” as used herein refers to a value that is within 10% of the stated number or range.

In some embodiments, the amiodarone, or an analog or derivative thereof, or a pharmaceutically acceptable salt, isotopically labeled compound, tautomer, stereoisomer, solvate, tautomer, hydrate, polymorph, co-crystal, or prodrug thereof, is administered prior to, concurrently with, or subsequent to the administration of metoprolol, or an analog or derivative thereof, or a pharmaceutically acceptable salt, isotopically labeled compound, tautomer, stereoisomer, solvate, tautomer, hydrate, polymorph, co-crystal, or prodrug thereof. In some embodiments, the amiodarone, or an analog or derivative thereof, or a pharmaceutically acceptable salt, isotopically labeled compound, tautomer, stereoisomer, solvate, tautomer, hydrate, polymorph, co-crystal, or prodrug thereof, is administered prior to, concurrently with, or subsequent to the cardiac graft. In some embodiments, the amiodarone, or an analog or derivative thereof, or a pharmaceutically acceptable salt, isotopically labeled compound, tautomer, stereoisomer, solvate, tautomer, hydrate, polymorph, co-crystal, or prodrug thereof, is administered prior to, concurrently with, or subsequent to the administration of ivabradine, or an analog or derivative thereof, or a pharmaceutically acceptable salt, isotopically labeled compound, tautomer, stereoisomer, solvate, tautomer, hydrate, polymorph, co-crystal, or prodrug thereof. In some embodiments, the amiodarone, or an analog or derivative thereof, or a pharmaceutically acceptable salt, isotopically labeled compound, tautomer, stereoisomer, solvate, tautomer, hydrate, polymorph, co-crystal, or prodrug thereof, is administered prior to, concurrently with, or subsequent to the administration of mexiletine, or an analog or derivative thereof, or a pharmaceutically acceptable salt, isotopically labeled compound, tautomer, stereoisomer, solvate, tautomer, hydrate, polymorph, co-crystal, or prodrug thereof. In some embodiments, the amiodarone, or an analog or derivative thereof, or a pharmaceutically acceptable salt, isotopically labeled compound, tautomer, stereoisomer, solvate, tautomer, hydrate, polymorph, co-crystal, or prodrug thereof, is administered prior to, concurrently with, or subsequent to the administration of verapamil, or an analog or derivative thereof, or a pharmaceutically acceptable salt, isotopically labeled compound, tautomer, stereoisomer, solvate, tautomer, hydrate, polymorph, co-crystal, or prodrug thereof.

In some embodiments, the metoprolol, or an analog or derivative thereof, or a pharmaceutically acceptable salt, isotopically labeled compound, tautomer, stereoisomer, solvate, tautomer, hydrate, polymorph, co-crystal, or prodrug thereof, is administered prior to, concurrently with, or subsequent to the administration of ivabradine, or an analog or derivative thereof, or a pharmaceutically acceptable salt, isotopically labeled compound, tautomer, stereoisomer, solvate, tautomer, hydrate, polymorph, co-crystal, or prodrug thereof. In some embodiments, the metoprolol, or an analog or derivative thereof, or a pharmaceutically acceptable salt, isotopically labeled compound, tautomer, stereoisomer, solvate, tautomer, hydrate, polymorph, co-crystal, or prodrug thereof, is administered prior to, concurrently with, or subsequent to the administration of the cardiac graft. In some embodiments, the metoprolol, or an analog or derivative thereof, or a pharmaceutically acceptable salt, isotopically labeled compound, tautomer, stereoisomer, solvate, tautomer, hydrate, polymorph, co-crystal, or prodrug thereof, is administered prior to, concurrently with, or subsequent to the administration of mexiletine, or an analog or derivative thereof, or a pharmaceutically acceptable salt, isotopically labeled compound, tautomer, stereoisomer, solvate, tautomer, hydrate, polymorph, co-crystal, or prodrug thereof. In some embodiments, the metoprolol, or an analog or derivative thereof, or a pharmaceutically acceptable salt, isotopically labeled compound, tautomer, stereoisomer, solvate, tautomer, hydrate, polymorph, co-crystal, or prodrug thereof, is administered prior to, concurrently with, or subsequent to the administration of verapamil, or an analog or derivative thereof, or a pharmaceutically acceptable salt, isotopically labeled compound, tautomer, stereoisomer, solvate, tautomer, hydrate, polymorph, co-crystal, or prodrug thereof.

In some embodiments, the ivabradine, or an analog or derivative thereof, or a pharmaceutically acceptable salt, isotopically labeled compound, tautomer, stereoisomer, solvate, tautomer, hydrate, polymorph, co-crystal, or prodrug thereof, is administered prior to, concurrently with, or subsequent to the administration of mexiletine, or an analog or derivative thereof, or a pharmaceutically acceptable salt, isotopically labeled compound, tautomer, stereoisomer, solvate, tautomer, hydrate, polymorph, co-crystal, or prodrug thereof. In some embodiments, the ivabradine, or an analog or derivative thereof, or a pharmaceutically acceptable salt, isotopically labeled compound, tautomer, stereoisomer, solvate, tautomer, hydrate, polymorph, co-crystal, or prodrug thereof, is administered prior to, concurrently with, or subsequent to the administration of verapamil, or an analog or derivative thereof, or a pharmaceutically acceptable salt, isotopically labeled compound, tautomer, stereoisomer, solvate, tautomer, hydrate, polymorph, co-crystal, or prodrug thereof. In some embodiments, the ivabradine, or an analog or derivative thereof, or a pharmaceutically acceptable salt, isotopically labeled compound, tautomer, stereoisomer, solvate, tautomer, hydrate, polymorph, co-crystal, or prodrug thereof, is administered prior to, concurrently with, or subsequent to the administration of the cardiac graft.

In some embodiments, the mexiletine, or an analog or derivative thereof, or a pharmaceutically acceptable salt, isotopically labeled compound, tautomer, stereoisomer, solvate, tautomer, hydrate, polymorph, co-crystal, or prodrug thereof, is administered prior to, concurrently with, or subsequent to the administration of the verapamil, or an analog or derivative thereof, or a pharmaceutically acceptable salt, isotopically labeled compound, tautomer, stereoisomer, solvate, tautomer, hydrate, polymorph, co-crystal, or prodrug thereof. In some embodiments, the mexiletine, or an analog or derivative thereof, or a pharmaceutically acceptable salt, isotopically labeled compound, tautomer, stereoisomer, solvate, tautomer, hydrate, polymorph, co-crystal, or prodrug thereof, is administered prior to, concurrently with, or subsequent to the administration of the cardiac graft.

In some embodiments, the verapamil, or an analog or derivative thereof, or a pharmaceutically acceptable salt, isotopically labeled compound, tautomer, stereoisomer, solvate, tautomer, hydrate, polymorph, co-crystal, or prodrug thereof, is administered prior to, concurrently with, or subsequent to the administration of the cardiac graft.

The pharmaceutical agents described herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In some embodiments, when multiple doses are administered to a subject, any two doses of the multiple doses include different or substantially the same amounts of a compound described herein. In some embodiments, when multiple doses are administered to a subject, the frequency of administering the multiple doses to the subject is three doses a day, two doses a day, one dose a day, one dose every other day, one dose every third day, one dose every week, one dose every two weeks, one dose every three weeks, or one dose every four weeks. In some embodiments, the frequency of administering the multiple doses to the subject is one dose per day. In some embodiments, the frequency of administering the multiple doses to the subject is two doses per day. In some embodiments, the frequency of administering the multiple doses to the subject is three doses per day. In some embodiments, when multiple doses are administered to a subject, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject or cell. In some embodiments, the duration between the first dose and last dose of the multiple doses is three months, six months, or one year. In some embodiments, the duration between the first dose and last dose of the multiple doses is the lifetime of the subject. In some embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a pharmaceutical agent described herein. In some embodiments, a dose described herein includes independently between 1 mg and 3 mg, inclusive, of a pharmaceutical agent described herein. In some embodiments, a dose described herein includes independently between 3 mg and 10 mg, inclusive, of a compound described herein.

In some embodiments, a pharmaceutical agent is a cardiac graft. In some embodiments, a pharmaceutical agent is a cardiomyocyte. In some embodiments, a pharmaceutical agent is an anti-arrhythmic agent.

The anti-arrhythmic agents described herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration.

In some embodiments, the dose of amiodarone, or an analog or derivative thereof, or a pharmaceutically acceptable salt, isotopically labeled compound, tautomer, stereoisomer, solvate, tautomer, hydrate, polymorph, co-crystal, or prodrug thereof, is 100-1400, 100-200, 200-400, 400-600, 600-800, 800-1000, 200-2000, 400-1200, 600-1000, 700-900, 750-850, about 800, 800-1600, 1000-1400, 1100-1300, or 1150-1250, inclusive, mg per day, or about 1200 mg per day. In some embodiments, the dose of amiodarone, or an analog or derivative thereof, or a pharmaceutically acceptable salt, isotopically labeled compound, tautomer, stereoisomer, solvate, tautomer, hydrate, polymorph, co-crystal, or prodrug thereof, is administered once a day, more than once a day, or twice a day. In some embodiments, the dose of amiodarone, or an analog or derivative thereof, or a pharmaceutically acceptable salt, isotopically labeled compound, tautomer, stereoisomer, solvate, tautomer, hydrate, polymorph, co-crystal, or prodrug thereof, is administered orally twice a day (e.g., a daily dose of 800 mg is administered in two separate doses of about 400 mg BID).

In some embodiments, the dose of metoprolol, or an analog or derivative thereof, or a pharmaceutically acceptable salt, isotopically labeled compound, tautomer, stereoisomer, solvate, tautomer, hydrate, polymorph, co-crystal, or prodrug thereof, is 50-150, 75-125, 90-110, or about 100 mg per day. In some embodiments, the daily dose of metoprolol, or an analog or derivative thereof, or a pharmaceutically acceptable salt, isotopically labeled compound, tautomer, stereoisomer, solvate, tautomer, hydrate, polymorph, co-crystal, or prodrug thereof, is administered once a day, more than once a day, twice a day, or orally twice a day (e.g., a daily dose of 100 mg is administered in two separate doses of about 50 mg BID).

In some embodiments, the dose of ivabradine, or an analog or derivative thereof, or a pharmaceutically acceptable salt, isotopically labeled compound, tautomer, stereoisomer, solvate, tautomer, hydrate, polymorph, co-crystal, or prodrug thereof, is 1-25, 5-15, 8-12, or about 10 mg per day. In some embodiments, the daily dose of ivabradine, or an analog or derivative thereof, or a pharmaceutically acceptable salt, isotopically labeled compound, tautomer, stereoisomer, solvate, tautomer, hydrate, polymorph, co-crystal, or prodrug thereof, is administered once a day, more than once a day, twice a day, or orally twice a day (e.g., a dose of 10 mg per day is administered in two separate doses of about 5 mg BID).

In some embodiments, the dose of mexiletine, or an analog or derivative thereof, or a pharmaceutically acceptable salt, isotopically labeled compound, tautomer, stereoisomer, solvate, tautomer, hydrate, polymorph, co-crystal, or prodrug thereof, is 100-600, 150-450, 200-400, about 200, about 225, about 250, about 275, about 300, about 325, about 350, about 375, or about 400 mg per day. In some embodiments, the dose of mexiletine, or an analog or derivative thereof, or a pharmaceutically acceptable salt, isotopically labeled compound, tautomer, stereoisomer, solvate, tautomer, hydrate, polymorph, co-crystal, or prodrug thereof, is administered once a day, more than once a day, twice a day, or orally twice a day (e.g., a dose of 200 mg per day is administered in two separate doses of about 100 mg BID).

In some embodiments, the dose of verapamil, or an analog or derivative thereof, or a pharmaceutically acceptable salt, isotopically labeled compound, tautomer, stereoisomer, solvate, tautomer, hydrate, polymorph, co-crystal, or prodrug thereof, is 50-200, 75-150, 100-150, or about 120 mg per day. In some embodiments, the dose of verapamil, or an analog or derivative thereof, or a pharmaceutically acceptable salt, isotopically labeled compound, tautomer, stereoisomer, solvate, tautomer, hydrate, polymorph, co-crystal, or prodrug thereof, is administered once a day, more than once a day, or twice a day.

In some embodiments, the dose of lidocaine, or an analog or derivative thereof, or a pharmaceutically acceptable salt, isotopically labeled compound, tautomer, stereoisomer, solvate, tautomer, hydrate, polymorph, co-crystal, or prodrug thereof, is about 0.1 to 1.5, about 0.5 to 1.0, or about 0.75 mg/min IV.

In some embodiments, the dose of procainamide, or an analog or derivative thereof, or a pharmaceutically acceptable salt, isotopically labeled compound, tautomer, stereoisomer, solvate, tautomer, hydrate, polymorph, co-crystal, or prodrug thereof, is about 0.1 to 1.5, about 0.5 to 1.0, or about 0.75 mg/min IV.

In some embodiments, the amiodarone, or an analog or derivative thereof, or a pharmaceutically acceptable salt, isotopically labeled compound, tautomer, stereoisomer, solvate, tautomer, hydrate, polymorph, co-crystal, or prodrug thereof, is administered parenterally, intravenously, and/or orally. In some embodiments, the amiodarone, or an analog or derivative thereof, or a pharmaceutically acceptable salt, isotopically labeled compound, tautomer, stereoisomer, solvate, tautomer, hydrate, polymorph, co-crystal, or prodrug thereof, is administered intravenously and orally.

In some embodiments, the metoprolol, or an analog or derivative thereof, or a pharmaceutically acceptable salt, isotopically labeled compound, tautomer, stereoisomer, solvate, tautomer, hydrate, polymorph, co-crystal, or prodrug thereof, is administered parenterally, intravenously, and/or orally. In some embodiments, the metoprolol, or an analog or derivative thereof, or a pharmaceutically acceptable salt, isotopically labeled compound, tautomer, stereoisomer, solvate, tautomer, hydrate, polymorph, co-crystal, or prodrug thereof, is administered intravenously and orally.

In some embodiments, the ivabradine, or an analog or derivative thereof, or a pharmaceutically acceptable salt, isotopically labeled compound, tautomer, stereoisomer, solvate, tautomer, hydrate, polymorph, co-crystal, or prodrug thereof, is administered parenterally, intravenously, and/or orally. In some embodiments, the ivabradine, or an analog or derivative thereof, or a pharmaceutically acceptable salt, isotopically labeled compound, tautomer, stereoisomer, solvate, tautomer, hydrate, polymorph, co-crystal, or prodrug thereof, is administered intravenously and orally.

In some embodiments, the mexiletine, or an analog or derivative thereof, or a pharmaceutically acceptable salt, isotopically labeled compound, tautomer, stereoisomer, solvate, tautomer, hydrate, polymorph, co-crystal, or prodrug thereof, is administered parenterally, intravenously, and/or orally. In some embodiments, the mexiletine, or an analog or derivative thereof, or a pharmaceutically acceptable salt, isotopically labeled compound, tautomer, stereoisomer, solvate, tautomer, hydrate, polymorph, co-crystal, or prodrug thereof, is administered intravenously and orally.

In some embodiments, the lidocaine, or an analog or derivative thereof, or a pharmaceutically acceptable salt, isotopically labeled compound, tautomer, stereoisomer, solvate, tautomer, hydrate, polymorph, co-crystal, or prodrug thereof, is administered parenterally, intravenously, and/or orally. In some embodiments, the lidocaine, or an analog or derivative thereof, or a pharmaceutically acceptable salt, isotopically labeled compound, tautomer, stereoisomer, solvate, tautomer, hydrate, polymorph, co-crystal, or prodrug thereof, is administered intravenously and orally.

In some embodiments, the procainamide, or an analog or derivative thereof, or a pharmaceutically acceptable salt, isotopically labeled compound, tautomer, stereoisomer, solvate, tautomer, hydrate, polymorph, co-crystal, or prodrug thereof, is administered parenterally, intravenously, and/or orally. In some embodiments, the procainamide, or an analog or derivative thereof, or a pharmaceutically acceptable salt, isotopically labeled compound, tautomer, stereoisomer, solvate, tautomer, hydrate, polymorph, co-crystal, or prodrug thereof, is administered intravenously and orally.

In some embodiments, the verapamil, or an analog or derivative thereof, or a pharmaceutically acceptable salt, isotopically labeled compound, tautomer, stereoisomer, solvate, tautomer, hydrate, polymorph, co-crystal, or prodrug thereof, is administered parenterally, intravenously, and/or orally. In some embodiments, the verapamil, or an analog or derivative thereof, or a pharmaceutically acceptable salt, isotopically labeled compound, tautomer, stereoisomer, solvate, tautomer, hydrate, polymorph, co-crystal, or prodrug thereof, is administered intravenously and orally.

In some embodiments, at least two or more of amiodarone, metoprolol, ivabradine, mexiletine, lidocaine, verapamil, and/or procainamide, optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug, are administered to the subject at least once per day for about 1-12, about 1-10, about 1-8, about 1-6, about 1-4, about 2-4, about 3-8, or about 4-10 weeks after the cardiac graft is administered.

In some embodiments, at least two or more of amiodarone, metoprolol, ivabradine, mexiletine, lidocaine, verapamil, and/or procainamide, optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug, are administered to the subject at least once per day for about 1-12, about 1-6, or about 1-3 months after the cardiac graft is administered.

In some embodiments, at least two or more of amiodarone, metoprolol, ivabradine, mexiletine, lidocaine, verapamil, and/or procainamide, optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug, are administered to the subject at least once per day until the arrhythmia has ceased. In some embodiments, at least two or more of amiodarone, metoprolol, ivabradine, mexiletine, lidocaine, verapamil, and/or procainamide, optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug, are administered to the subject to maintain a resting heart rate of less than or equal to 150 beats per minute (bpm). In some embodiments, at least two or more of amiodarone, metoprolol, ivabradine, mexiletine, lidocaine, verapamil, and/or procainamide, optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug, are administered to the subject at least once per day to maintain a resting heart rate of less than or equal to 100 beats per minute (bpm). In some embodiments, at least two or more of amiodarone, metoprolol, ivabradine, mexiletine, lidocaine, verapamil, and/or procainamide, optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug, are administered to the subject at least once per day to maintain a resting heart rate of 60 to 150 bpm, e.g., 60-80 bpm, 50-90 bpm, 60-140 bpm, 60-130 bpm, 60-120 bpm, 60-110 bpm, 60-100 bpm, 60-90 bpm or 60-80 bpm.

In some embodiments, the anti-arrhythmic agents used in this disclosure are as shown in Table 1.

TABLE 1
List of anti-arrhythmic agents used in this disclosure

| Name | Drug I.D. No.: |
|---|---|
| Amiodarone | DIN 02243836 |
| Amiodarone | DIN 02245248 |
| Metoprolol | DIN 00618632 |
| Ivabradine | DIN 02459973 |
| Mexiletine | DIN 02230360 |
| Lidocaine | DIN 02422026 |
| Procainamide | DIN 02184486 |
| Verapamil | DIN 02166739 |

In some embodiments, the method further comprises administering cardiomyocytes to the subject in need thereof. In some embodiments, the method comprises administering the cardiomyocytes to the subject in an amount effective to treat a cardiovascular disease, disorder, or symptom thereof. In some embodiments, the effective amount of cardiomyocytes is an amount effective to treat or prevent a cardiomyopathy. In some embodiments, the effective amount of cardiomyocytes is an amount effective to treat heart failure.

In some embodiments, a subject to which a cardiomyocyte graft is to be administered is administered one or more immunosuppressants. In some embodiments, administration of immunosuppressants. In some embodiments, the method further comprises administering to the subject in need thereof one or more immunosuppressants. In some embodiments, the method further comprises administering to the subject in need thereof two or more immunosuppressants. In some embodiments, the method further comprises administering to the subject in need thereof three or more immunosuppressants. In some embodiments, the method further comprises administering to the subject in need thereof one, two, three, or four immunosuppressants. In some embodiments, at least one of the immunosuppressants is a glucocorticoid (e.g., cortisol, cortisone, prednisone, prednisolone, methylprednisolone, dexamethasone, betamethasone, triamcinolone, deflazacort, fludrocortisone acetate, deoxycorticosterone acetate, aldosterone, or beclomethasone, or a pharmaceutically acceptable salt, isotopically labeled compound, tautomer, stereoisomer, solvate, tautomer, hydrate, polymorph, co-crystal, or prodrug thereof). In some embodiments, at least one of the immunosuppressants is a cytostatic (e.g., an alkylating agent, antimetabolite, cytotoxic antibiotic, monoclonal antibody, polyclonal antibody, T-cell receptor directed antibody, or IL-2 receptor directed antibody; or methotrexate, azathioprine, or mercaptopurine, or a pharmaceutically acceptable salt, isotopically labeled compound, tautomer, stereoisomer, solvate, tautomer, hydrate, polymorph, co-crystal, or prodrug thereof). In some embodiments, at least one of the immunosuppressants is a drug acting on immunophilins (e.g., cyclosporin A, tacrolimus, sirolimus, everolimus, or zotarolimus, or a pharmaceutically acceptable salt, isotopically labeled compound, tautomer, stereoisomer, solvate, tautomer, hydrate, polymorph, co-crystal, or prodrug thereof). In some embodiments, at least one of the immunosuppressants is an interferon, opioid, TNF binding protein, mycophenolate, fingolimod, or myriocin, or a pharmaceutically acceptable salt, isotopically labeled compound, tautomer, stereoisomer, solvate, tautomer, hydrate, polymorph, co-crystal, or prodrug thereof. In some embodiments, at least one of the immunosuppressants is cyclosporin A, methylprednisolone, or abatacept, or a pharmaceutically acceptable salt, isotopically labeled compound, tautomer, stereoisomer, solvate, tautomer, hydrate, polymorph, co-crystal, or prodrug thereof.

In some embodiments, at least one of the immunosuppressants is administered prior to, e.g., between 1 and 3 hours, between 3 and 6 hours, between 6 and 12 hours, between 12 and 24 hours, between 1 and 2 days, between 2 and 3 days, between 3 and 5 days, between 5 and 7 days, between 7 and 10 days, or between 10 and 14 days, inclusive, prior to the administration of the cardiac graft. In some embodiments, at least one of the immunosuppressants is administered substantially concurrently with the administration of the cardiac graft. In some embodiments, at least one of the immunosuppressants is administered subsequent to, e.g., between 1 and 3 hours, between 3 and 6 hours, between 6 and 12 hours, between 12 and 24 hours, between 1 and 2 days, between 2 and 4 days, between 4 and 7 days, between 1 and 2 weeks, between 2 and 4 weeks, or between 1 and 2 months, inclusive, subsequent to the administration of the cardiac graft.

In some embodiments, at least one of the immunosuppressants is administered one, two, three, or four times a day.

In some embodiments, the method further comprises one or more additional administrations of at least one of the immunosuppressants.

In some embodiments, the total number of the administrations of the immunosuppressants is between 1 and 3, between 3 and 10, between 10 and 30, between 30 and 100, between 100 and 300, or between 300 and 1000, inclusive, or greater than 1000.

In some embodiments, the time duration between the first and last administrations of the immunosuppressants is between 1 and 3 days, between 3 and 7 days, between 1 and 2 weeks, between 2 and 4 weeks, between 1 and 3 months, between 3 and 6 months, between 6 and 12 months, between 1 and 2 years, between 2 and 5 years, or between 5 and 10 years, inclusive, or the lifetime of the subject.

In some embodiments, at least one of the immunosuppressants is administered according to a method described in Romagnuolo et al., Stem Cell Reports, 2019, 12, 967-981, incorporated herein by reference in its entirety.

In some embodiments, cyclosporin A is administered: (1) orally; (2) twice a day; (3) at a dose of 3.6 to 36 mg/kg, e.g., 7.1 to 11 mg/kg, inclusive; (4) 5±30% days prior to the administration of the cardiac graft; and/or (5) for as long as at least one other anti-arrhythmic agent described herein is administered.

In some embodiments, methylprednisolone is administered: (1) orally; (2) at a single dose of 179±30% mg/kg on the day of the administration of the cardiac graft; and/or (3) at single tapered doses from 179±30% mg/kg at the first day after the administration of the cardiac graft to 89±30% mg/kg per day over 14±30% days, and at 89±30% mg/kg per day thereafter for as long as at least one other anti-arrhythmic agent described herein is administered.

In some embodiments, abatacept is administered: (1) intravenously; (2) at a single dose of 8.9±30% mg/kg on the day of the administration of the cardiac graft; and/or (3) at single doses of 8.9±30% mg/kg every 14±30% days subsequent to the day of the administration of the cardiac graft for as long as at least one other anti-arrhythmic agent described herein is administered.

When administered to a subject, effective amounts of the pharmaceutical agent will depend on the particular disease being treated; the severity of the disease; individual patient parameters including age, physical condition, size and weight, concurrent treatment, frequency of treatment, and the mode of administration. These factors are well known to those of ordinary skill in the art and can be addressed with no more than routine experimentation. In some embodiments, a maximum dose is used, that is, the highest safe dose according to sound medical judgment.

An effective amount of a compound typically will vary from about 0.001 mg/kg to about 2000 mg/kg in one or more dose administrations, for one or several days (depending of course of the mode of administration and the factors discussed above).

Actual dosage levels of the pharmaceutical agent can be varied to obtain an amount that is effective to achieve the desired therapeutic response for a particular patient, compositions, and mode of administration. The selected dosage level depends upon the activity of the particular compound, the route of administration, the tissue being treated, and prior medical history of the patient being treated. However, it is within the skill of the art to start doses of the compound at levels lower than required to achieve the desired therapeutic effort and to gradually increase the dosage until the desired effect is achieved

In some embodiments, the pharmaceutically acceptable salt is suitable for use in contact with the tissues of humans and lower animals without undue toxicity, irritation, allergic response and the like, and are commensurate with a reasonable benefit/risk ratio. In some embodiments, the pharmaceutically acceptable salt is as described in Berge et al., describe pharmaceutically acceptable salts in detail in J. Pharmaceutical Sciences, 1977, 66, 1-19, incorporated herein by reference. In some embodiments, the pharmaceutically acceptable salt is derived from suitable inorganic and organic acids and bases. In some embodiments, the pharmaceutically acceptable salt is a salt of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. In some embodiments, the pharmaceutically acceptable salt is adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, or valerate salt. In some embodiments, the pharmaceutically acceptable salt is a salt derived from an appropriate base. In some embodiments, the pharmaceutically acceptable salt is an alkali metal, alkaline earth metal, ammonium, or N⁺(C₁₋₄ alkyl)₄⁻ salt. In some embodiments, the pharmaceutically acceptable salt is a sodium, lithium, potassium, calcium, or magnesium salt. In some embodiments, the pharmaceutically acceptable salt is a nontoxic ammonium, quaternary ammonium, or amine salt formed using counterions such as halide, hydroxide, carboxylate, sulfate, phosphate, nitrate, lower alkyl sulfonate, and aryl sulfonate.

In some embodiments, the solvate is a form of a pharmaceutical agent, or a salt thereof, that are associated with a solvent, usually by a solvolysis reaction. This physical association may include hydrogen bonding. In some embodiments, the solvent is water, methanol, ethanol, acetic acid, DMSO, THF, or diethyl ether. In some embodiments, the solvate is a pharmaceutically acceptable solvate. In some embodiments, the solvate further includes both stoichiometric solvates and non-stoichiometric solvates. In some embodiments, the solvate is capable of isolation, for example, when one or more solvent molecules are incorporated in the crystal lattice of a crystalline solid. Solvates may encompasses both solution-phase and isolable solvates. In some embodiments, the solvate is a hydrate, ethanolate, or methanolate.

In some embodiments, the hydrate is a pharmaceutical agent that is associated with water. In some embodiments, the number of the water molecules contained in a hydrate of a pharmaceutical agent is in a definite ratio to the number of the pharmaceutical agent molecules in the hydrate. In some embodiments, a hydrate of a pharmaceutical agent is by the general formula R·x H₂O, wherein R is the pharmaceutical agent and wherein x is a number greater than 0. In some embodiments, the hydrate is a monohydrate (x is 1), lower hydrate (x is a number greater than 0 and smaller than 1, e.g., hemihydrate (R·0.5 H₂O)), or polyhydrate (x is a number greater than 1, e.g., dihydrate (R·2 H₂O) or hexahydrate (R·6 H₂O)).

In some embodiments, the tautomer is a pharmaceutical agent that may be in interchangeable structural forms, and that vary in the displacement of hydrogen atoms and electrons. In some embodiments, two structures are in equilibrium through the movement of π electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base. Tautomeric forms may be relevant to the attainment of the optimal chemical reactivity and biological activity of a pharmaceutical agent. The exact ratio of the tautomers may depend on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

In some embodiments, an isomer is a pharmaceutical agent that has the same molecular formula but differ in the nature or sequence of bonding of their atoms or the arrangement of their atoms in space as compared to another pharmaceutical agent. In some embodiments, a stereoisomer is an isomer that differs in the arrangement of its atoms in space as compared to another isomer. In some embodiments, a stereoisomer is a diastereomer or enantiomer. When a pharmaceutical agent has an asymmetric center, for example, it is bonded to four different groups, a pair of enantiomers is possible. An enantiomer may be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral pharmaceutical agent may exist as either individual enantiomer or as a mixture thereof. In some embodiments, a racemic mixture is a mixture containing equal proportions of the enantiomers.

In some embodiments, the crystalline form is a solid form substantially exhibiting three-dimensional order. In some embodiments, a crystalline form of a solid is a solid form that is substantially not amorphous. In some embodiments, the X-ray powder diffraction (XRPD) pattern of a crystalline form includes one or more sharply defined peaks.

In some embodiments, the amorphous form is a solid form substantially lacking three-dimensional order. In some embodiments, an amorphous form of a solid is a solid form that is substantially not crystalline. In some embodiments, the X-ray powder diffraction (XRPD) pattern of an amorphous form includes a wide scattering band with a peak at 2θ of, e.g., between 2° and 70°, inclusive, using CuKα radiation. In some embodiments, the XRPD pattern of an amorphous form further includes one or more peaks attributed to crystalline structures. In some embodiments, the maximum intensity of any one of the one or more peaks attributed to crystalline structures observed at a 2θ of between 2° and 70°, inclusive, is not more than 300-fold, not more than 100-fold, not more than 30-fold, not more than 10-fold, or not more than 3-fold of the maximum intensity of the wide scattering band. In some embodiments, the XRPD pattern of an amorphous form includes no peaks attributed to crystalline structures.

In some embodiments, the co-crystal is a crystalline structure comprising at least two different components (e.g., a pharmaceutical agent disclosed herein and an acid), wherein each of the components is independently an atom, ion, or molecule. In some embodiments, none of the components is a solvent. In some embodiments, at least one of the components is a solvent. A co-crystal of a pharmaceutical agent disclosed herein and an acid is different from a salt formed from a pharmaceutical agent disclosed herein and the acid. In the salt, a pharmaceutical agent disclosed herein is complexed with the acid in a way that proton transfer (e.g., a complete proton transfer) from the acid to a pharmaceutical agent disclosed herein easily occurs at room temperature. In the co-crystal, however, a pharmaceutical agent disclosed herein is complexed with the acid in a way that proton transfer from the acid to a pharmaceutical agent disclosed herein does not easily occur at room temperature. In some embodiments, in the co-crystal, there is no proton transfer from the acid to a pharmaceutical agent disclosed herein. In some embodiments, in the co-crystal, there is partial proton transfer from the acid to a pharmaceutical agent disclosed herein. Co-crystals may be useful to improve the properties (e.g., solubility, stability, and ease of formulation) of a pharmaceutical agent disclosed herein.

In some embodiments, the polymorph is a crystalline form of a pharmaceutical agent (or a salt, hydrate, or solvate thereof) in a particular crystal packing arrangement. In some embodiments, all polymorphs have the same elemental composition. In some embodiments, different crystalline forms have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a pharmaceutical agent may be prepared by crystallization under different conditions.

In some embodiments, the prodrug is a pharmaceutical agent that has cleavable groups and becomes chemically active by solvolysis or under physiological conditions. In some embodiments, the prodrug is an ester derivative. In some embodiments, the prodrug is acid sensitive. In some embodiments, the acid sensitive prodrug is advantages of solubility, tissue compatibility, or delayed release in the mammalian organism. In some embodiments, the prodrug is as described in Bundgard, H., Design of Prodrugs, pp. 7-9, 21-24, Elsevier, Amsterdam 1985, incorporated herein by reference in its entirety. In some embodiments, the prodrug is an acid derivative, such as, ester prepared by reaction of the parent acid with a suitable alcohol, or amide prepared by reaction of the parent acid pharmaceutical agent with a substituted or unsubstituted amine, or acid anhydride, or mixed anhydride. In some embodiments, the prodrug is an aliphatic or aromatic ester, amide, or anhydride derived from acidic group pendant on the pharmaceutical agent. In some embodiments, the prodrug is a double ester prodrug, such as an (acyloxy)alkyl ester or ((alkoxycarbonyl)oxy)alkylester.

In some embodiments, the subject is a human (e.g., a male or female of any age group, e.g., a pediatric subject (e.g., infant, child, adolescent) or adult subject (e.g., young adult, middle-aged adult, or senior adult)). In some embodiments, the subject is a non-human animal. In some embodiments, the subject is any one of mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). The animal may be a male or female and at any stage of development. A non-human animal may be a transgenic animal. In some embodiments, the subject is based at least in part on the criteria below:
(1) Relative risk reduction in the rate of cardiac mortality;
(2) Relative risk reduction in the rate of heart failure hospitalization;
(3) Reduction in the rate of Major Averse Cardiac Events (MACE), a composite endpoint of CV death, non-fatal myocardial infarction, and non-fatal stroke;
(4) Improvement in functional capacity including improvement in 6-minute walk distance (6MWD) demonstrated in a double blinded study, improvement in measures of daily activity (e.g. accelerometry data);
(5) Improvement in symptoms or a composite symptom score/quality of life (QoL) measures such as the Minnesota Living with Heart Failure questionnaire (MLHFQ);
(6) Improvement in cardiopulmonary testing;
(7) Improvement in clinical biomarker profile including left ventricular ejection fraction and left ventricular dimensions (suggestive or reverse remodeling);
(8) Freedom from cardiac transplantation or mechanical support;
(9) Improvement in weaning from LVAD or other mechanical support; and/or
(10) Other symptoms, such as dyspnea, fluid retention, and/or edema.

In some embodiments, the subject is based at least in part on the ejection fraction. In some embodiments, the subject has severe reduction in EF (e.g., EF<30%). In some embodiments, the subject has moderate reduction (EF<40%). In some embodiments, a subject is based for any one of the treatments described herein, on the basis of needing to improve any one or more of the following criteria:
(1) Relative risk reduction in the rate of cardiac mortality;
(2) Relative risk reduction in the rate of heart failure hospitalization;
(3) Reduction in the rate of Major Averse Cardiac Events (MACE), a composite endpoint of CV death, non-fatal myocardial infarction, and non-fatal stroke;
(4) Improvement in functional capacity including improvement in 6-minute walk distance (6MWD) demonstrated in a double blinded study, improvement in measures of daily activity (e.g. accelerometry data);
(5) Improvement in symptoms or a composite symptom score/quality of life (QoL) measures such as the Minnesota Living with Heart Failure questionnaire (MLHFQ);
(6) Improvement in cardiopulmonary testing;
(7) Improvement in clinical biomarker profile including left ventricular ejection fraction and left ventricular dimensions (suggestive or reverse remodeling);
(8) Freedom from cardiac transplantation or mechanical support;
(9) Improvement in weaning from LVAD or other mechanical support; and/or
(10) Other symptoms, such as dyspnea, fluid retention, and/or edema.

In some embodiments, the method does not comprise administering amiodarone and ivabradine, optionally each of which independently comprises a pharmaceutically acceptable salt, isotopically labeled compound, tautomer, stereoisomer, solvate, tautomer, hydrate, polymorph, co-crystal, or prodrug thereof. In some embodiments, the method does not comprise administering amiodarone and ivabradine. In certain embodiments, the subject has not been administered (e.g., between 1 and 2 days, between 2 and 4 days, between 4 and 7 days, between 1 and 2 weeks, between 2 and 4 weeks, between 1 and 2 months, or between 2 and 4 months, inclusive, prior to the administering step provided herein) amiodarone, metoprolol, ivabradine, mexiletine, lidocaine, verapamil, and/or procainamide, optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof.

In some embodiments, the administering or administration is implanting, absorbing, ingesting, injecting, inhaling, or otherwise introducing a pharmaceutical agent or pharmaceutical composition.

In some embodiments, the treatment or treating is reversing, alleviating, delaying the onset of, or inhibiting the progress of a pathological condition (e.g., a disease, disorder, or condition, or one or more signs or symptoms thereof). In some embodiments, a pharmaceutical agent or pharmaceutical composition is administered after one or more signs or symptoms have developed or have been observed. In some embodiments, a pharmaceutical agent or pharmaceutical composition is administered in the absence of signs or symptoms of the disease or condition. In some embodiments, a pharmaceutical agent or pharmaceutical composition is administered to a subject prior to the onset of symptoms (e.g., in light of a history of symptoms and/or in light of genetic or other susceptibility factors). In some embodiments, the treatment is continued after symptoms have resolved, for example, to delay or prevent recurrence. In some embodiments, the treatment is therapeutic treatment.

The terms “condition,” “disease,” and “disorder” are used interchangeably.

In some embodiments, the effective amount of a pharmaceutical agent is an amount sufficient to elicit the desired biological response, e.g., treating the condition. The effective amount may vary depending on such factors as the desired biological endpoint, the pharmacokinetics of the pharmaceutical agent, the condition being treated, the mode of administration, and the age, size, weight, and health of the subject.

In some embodiments, the therapeutically effective amount of a pharmaceutical agent is an amount sufficient to provide a therapeutic benefit in the treatment of a condition or to delay or minimize one or more symptoms associated with the condition. In some embodiments, the therapeutically effective amount is an amount of the pharmaceutical agent, alone or in combination with other therapies, which provides a therapeutic benefit in the treatment of the condition. In some embodiments, the therapeutically effective amount is an amount that improves overall therapy, reduces, delays, or avoids signs, symptoms or causes of the condition, or enhances the therapeutic efficacy of another pharmaceutical agent.

In some embodiments, the prophylactically effective amount of a pharmaceutical agent is an amount sufficient to prevent a condition, or one or more symptoms associated with the condition or prevent its recurrence. In some embodiments, the prophylactically effective amount is an amount of a pharmaceutical agent, alone or in combination with other agents, which provides a prophylactic benefit in the prevention of the condition. In some embodiments, the prophylactically effective amount is an amount that improves overall prophylaxis or enhances the prophylactic efficacy of another pharmaceutical agent.

In some embodiments, the effective amount is based at least in part on the criteria below:
(1) Relative risk reduction in the rate of cardiac mortality;
(2) Relative risk reduction in the rate of heart failure hospitalization;
(3) Reduction in the rate of Major Averse Cardiac Events (MACE), a composite endpoint of CV death, non-fatal myocardial infarction, and non-fatal stroke;
(4) Improvement in functional capacity including improvement in 6-minute walk distance (6MWD) demonstrated in a double blinded study, improvement in measures of daily activity (e.g. accelerometry data);
(5) Improvement in symptoms or a composite symptom score/quality of life (QoL) measures such as the Minnesota Living with Heart Failure questionnaire (MLHFQ);
(6) Improvement in cardiopulmonary testing;
(7) Improvement in clinical biomarker profile including left ventricular ejection fraction and left ventricular dimensions (suggestive or reverse remodeling);
(8) Freedom from cardiac transplantation or mechanical support;
(9) Improvement in weaning from LVAD or other mechanical support; and/or
(10) Other symptoms, such as dyspnea, fluid retention, and/or edema.

In some embodiments, the effective amount is based at least in part on the ejection fraction. In some embodiments, the effective amount results in >5% (e.g., >5%, >8%, >10%, >12%, >15%, >20%, >25%, or >30% or more) increase in EF.

In some embodiments, the cardiomyopathy is a disease of the heart muscle that may affect the heart's ability to pump blood. In some embodiments, the cardiomyopathy is dilated cardiomyopathy (e.g., alcoholic cardiomyopathy, congestive cardiomyopathy, diabetic cardiomyopathy, familial dilated cardiomyopathy, idiopathic cardiomyopathy, ischemic cardiomyopathy or coronary heart disease, peripartum cardiomyopathy, or primary cardiomyopathy), hypertrophic cardiomyopathy (e.g., obstructive or nonobstructive hypertrophic cardiomyopathy), restrictive cardiomyopathy (e.g., idiopathic or genetic restrictive cardiomyopathy), infiltrative cardiomyopathy (including cardiac amyloidosis, carcinoid heart disease, hemochromatosis, or sarcoidosis), arrhythmogenic (e.g., right ventricular dysplasia)), transthyretin amyloid cardiomyopathy (e.g., hereditary or wild-type transthyretin amyloid cardiomyopathy, ischemic heart disease, myocardial infarction (acute and chronic), left ventricular heart failure, right ventricular heart failure, myocarditis (e.g., myocarditis caused by bacterial or viral infection), cardiomyopathy caused by chronic infection including viral infection (such as HIV), bacterial infection (such as Lyme's disease) or parasitic infection (such as Chagas disease). In some embodiments, the heart disease is related to congenital heart disease (including hypoplastic left heart syndrome (HLHS), hypoplastic right heart syndrome (HRHS), or other cardiac birth defect. In some embodiments, the heart failure is left-sided heart failure, a right-sided heart failure, a diastolic heart failure, or a systolic heart failure. In some embodiments, the heart failure is congestive heart failure. In some embodiments the heart failure is heart failure with reduced ejection fraction (HFrEF). In some embodiments the heart failure is heart failure with borderline ejection fraction (HFbEF). In some embodiments the heart failure is heart failure with preserved ejection fraction (HFpEF). In some embodiments, the subject is suffering from one or more previous myocardial infarctions. In further embodiments, the one or more myocardial infarctions are in the ventricle (e.g., left ventricle) of the subject. Cardiac grafts as described herein results in replacement or repair of cardiac muscle and restoration of cardiac function in the subject, thus treating the cardiomyopathy.

In some embodiments, the immature cardiomyocyte is a cardiomyocyte derived from stem cells that is in various stages of differentiation to mature cardiomyocytes, e.g., progenitor cells, that may represent cardiac cells in early stages of fetal development. Immature cardiomyocytes as described herein, may have the following characteristics: (1) CD36 negative/low (relative to a mature cell), MLC2v negative/low (relative to a mature cell), and MLC2a positive/high (relative to a mature cell), e.g., using flow cytometric analysis; and (2) a spontaneous beating rate of between 0.4 and 0.6 Hz and a conduction velocity of 0.15-0.25 mm/ms using microelectrode array (MEA). In some embodiments, the immature cardiomyocyte is determined with NKX2-5+ as a marker.

In some embodiments, the mature cardiomyocyte is an immature cardiomyocyte derived from stem cells that has been further differentiated and has the following characteristics: (1) CD36 positive, MLC2v positive/high, and downregulated MLC2a as compared to immature cardiomyocytes (e.g., using flow cytometric analysis); and (2) a spontaneous beating rate of under 0.4 Hz and a conduction velocity of more than 0.25 mm/ms (e.g., 0.25-0.40 mm/ms) using microelectrode array (MEA). In some embodiments, mature cardiomyocytes are TNI positive/high. In some embodiments, the cardiomyocytes are matured in vivo. In some embodiments, the cardiomyocytes are matured in vitro. Non-limiting examples of cardiomyocyte differentiation and maturation procedures can be found in PCT Publication No.: WO 2014/200339, PCT Publication No.: WO 2017/039445, PCT Publication No.: WO 2020/227232, U.S. Publication No.: US 2020/0407687, each of which are incorporated herein by reference. In some embodiments, the mature cardiomyocyte is determined with NKX2-5+ as a marker.

In some embodiments, the cardiomyocytes are a mixture of mature cardiomyocytes and immature cardiomyocytes.

### Pharmaceutical Compositions

In another aspect, the present disclosure provides a pharmaceutical composition comprising two or more of:
amiodarone,
metoprolol,
ivabradine,
mexiletine,
lidocaine,
verapamil, and
procainamide,
optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof; and
optionally a pharmaceutically acceptable excipient.

In some embodiments, a pharmaceutical composition comprises two or more of:
amiodarone,
metoprolol,
ivabradine, and
mexiletine,
optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof.

In some embodiments, a pharmaceutical composition comprises:
amiodarone,
metoprolol, and
ivabradine,
optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof.

In some embodiments, a pharmaceutical composition comprises:
amiodarone, and
ivabradine,
optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof.

In some embodiments, a pharmaceutical composition comprises:
amiodarone,
metoprolol,
verapamil,
ivabradine,
lidocaine, and
procainamide,
optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof.

In some embodiments, a pharmaceutical composition comprises two or more of:
metoprolol,
ivabradine, and/or
mexiletine,
optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof.

In some embodiments, a pharmaceutical composition comprises two or more of:
amiodarone,
metoprolol, and/or
mexiletine,
optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof.

Pharmaceutical preparations and compounds are administered to a subject by any suitable route. For example, compositions can be administered orally, including sublingually, rectally, parenterally, intracisternally, intravaginally, intraperitoneally, topically and transdermally (as by powders, ointments, or drops), bucally, or nasally. The pharmaceutical preparations of the present disclosure may include or be diluted into a pharmaceutically-acceptable carrier. The term “pharmaceutically-acceptable carrier” as used herein means one or more compatible fillers, diluents or other such substances, which are suitable for administration to a human or other mammal such as a dog, cat, or horse. The term “carrier” denotes an organic or inorganic ingredient, natural or synthetic, with which the active ingredient is combined to facilitate the application. The carriers are capable of being commingled with the preparations of the present disclosure, and with each other, in a manner such that there is no interaction which would substantially impair the desired pharmaceutical efficacy or stability. Carriers suitable for oral, subcutaneous, intravenous, intramuscular, etc. formulations can be found in Remington's Pharmaceutical Sciences, Mack Publishing Company, Easton, Pa.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

The pharmaceutical agents described herein may be administered together with each other in a single dose or administered separately in different doses. The particular combination to employ in a regimen will take into account compatibility of the pharmaceutical agents described herein and/or the desired therapeutic and/or prophylactic effect to be achieved. In general, it is expected that the pharmaceutical agent(s) in combination will be utilized at levels that do not exceed the levels at which they are utilized individually. In some embodiments, the levels utilized in combination will be lower than those utilized individually.

Pharmaceutical compositions described herein can be prepared by any method known in the art of pharmacology. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj R 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In some embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In some embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

### Kits

Also encompassed by the present disclosure are kits (e.g., pharmaceutical packs). In some embodiments, the kit comprises (1) two or more of amiodarone, metoprolol, ivabradine, and/or mexiletine, optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug; and (2) instructions for administering (1) to treat or prevent arrhythmia in a subject in need thereof. In some embodiments, the kit further comprises (3) lidocaine or procainamide, optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug; and (4) instructions for administering (3) to treat or prevent arrhythmia in a subject in need thereof. In some embodiments, the subject in need thereof is in need of or has received a cardiac graft. In some embodiments, the instructions comprise information required by a regulatory agency, such as the U.S. Food and Drug Administration (FDA) or the European Agency for the Evaluation of Medicinal Products (EMA). In some embodiments, the instructions comprise prescribing information.

The present disclosure is further illustrated by the following Examples, which in no way should be construed as further limiting. The entire contents of all of the references (including literature references, issued patents, published patent applications, and co pending patent applications) cited throughout this application are hereby expressly incorporated by reference.

### EXAMPLES

### Example 1. Treatment of Graft-Induced Arrhythmia in Pig Model

Experiments were conducted to measure the effect of different combinations of anti-arrhythmic agents in pigs subjected to myocardial infarction (to mimic injury) and a graft (to treat cardiomyopathy resulting from the injury) comprising either mature or immature cardiomyocytes.

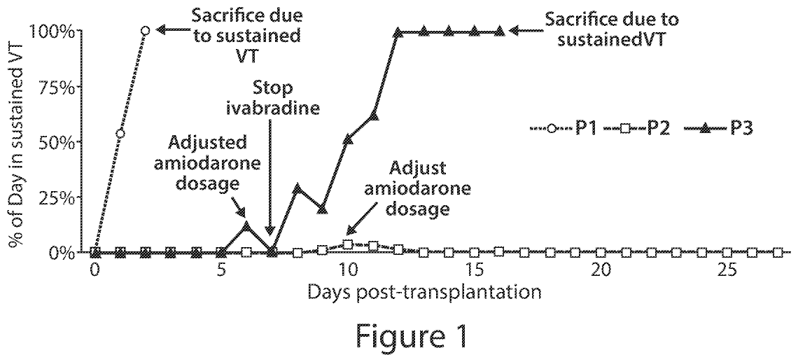

Cardiomyocytes were delivered directly into the scar region through transepicardial injections in swine 3-weeks post myocardial infarction (MI). Severe graft-related arrhythmias (heart rate >250 bpm) were observed following delivery of immature cardiomyocytes without any treatment with any anti-arrhythmic drugs (Pig ID, P1; FIG. 1). P1 had to be sacrificed within 48 hours post cell delivery due to continuous, sustained ventricular tachyarrhythmia. Table 2 illustrates the general treatment plan used to test whether the graft related arrhythmias can be reduced following transplantation of immature cardiomyocytes, where adjustment in dosages were done on a pig-by-pig basis based on overall response. For example, if non-sustained VT is observed, increase amiodarone dose from 600 to 1200 mg BID or discontinue amiodarone PO and start on constant rate infusion (CRI) of amiodarone at 0.75-1.5 mg/min. The anti-arrhythmic agents used in the examples are as shown in the table below:

| Anti-arrhythmic agent | | Drug Identification |
|---|---|---|
| generic name and strength | Salt form | Number |
| Amiodarone PO 200 mg | hydrochloride | DIN 02243836 |
| Amiodarone IV 50 mg/mL | hydrochloride | DIN 02245248 |
| Metoprolol 50 mg | hemitartrate | DIN 00618632 |
| Ivabradine 5 mg | hydrochloride | DIN 02459973 |
| Mexiletine 200 mg | hydrochloride | DIN 02230360 |
| Lidocaine 2% | hydrochloride | DIN 02422026 |
| Procainamide 100 mg/mL | hydrochloride | DIN 02184486 |
| Verapamil 120 mg | hydrochloride | DIN 02166739 |

TABLE 2
Generic anti-arrhythmic treatment
plan for immature cardiomyocytes

| | Pre-treatment | 1 day prior |
|---|---|---|
| | (4-wks prior | to cell therapy |
| Drug name | to cell therapy) | and onwards |
| Amiodarone | 400 mg BID | 600 mg BID |
| Metoprolol | | 50 mg BID |
| Verapamil | | 120 mg SID |
| Ivabradine | | 5 mg BID |
| Lidocaine | | 0.75 mg/min IV |
| Procainamide | | 0.75 mg/min IV |

Using the treatment plans denoted in FIG. 1, the incidences of graft related arrhythmia (Pig ID P2; FIG. 1) were successfully reduced. P2 spent majority of the days without any sustained VT (23/27 days). The highest incidence of VT occurred on day 10/11 post-cell therapy (<4% of the day spent in sustained VT), where adjusting the amiodarone dosage was effective at achieving normal sinus rhythm (by day 13 onwards).

For Pig ID P3, treatment plan was identical to P2. However, on day 7 post-transplantation (when pig was in normal sinus rhythm), ivabradine was removed from treatment plan. Within 24-hrs of removing ivabradine from the treatment regimen, P3 began to have sustained VT and by day 12, spent >95% of the day in sustained VT (FIG. 1).

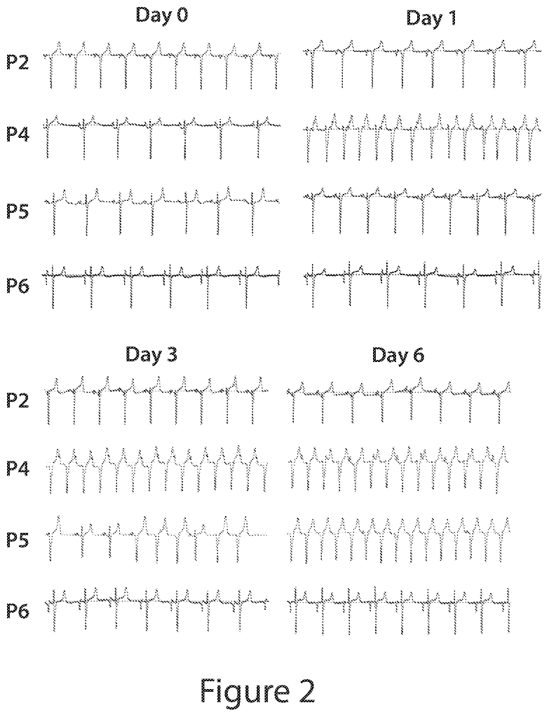

Removal of either procainamide (pig ID P4) or lidocaine (pig ID P5) from the 6-drug cocktail in Table 2 at day 0 results in sustained VT (FIG. 2). In addition, it was found that in pigs receiving mature cardiomyocytes, treatment with to the combination of anti-arrhythmics as shown in Table 3 resulted in prevention of graft related arrhythmias (See pig ID P6 in FIG. 2). The cocktail used when transplanting mature CMs is found in Table 3.

TABLE 3
Generic anti-arrhythmic treatment plan for mature cardiomyocytes

| Anti- | Pre-treatment | 1 day prior |
|---|---|---|
| arrhythmic | (4-wks prior to | to cell therapy |
| agent | cell therapy) | and onwards |
| Amiodarone | 600-800 mg BID | 1 g BID |
| Metoprolol | 25-50 mg BID | 50 mg BID |
| Ivabradine | | 5-15 mg BID |
| Mexiletine | | 100-200 mg BID |

TABLE 4
Summary of pigs treated with anti-arrhythmic
agents and severity of VT

| Pig | | Maturity of | Severe VT |
|---|---|---|---|
| ID | Treatment plan | cardiomyocyte | observed |
| P1 | No treatment | Immature | Yes |
| P2 | Amiodarone, metoprolol, | Immature | No |
| | ivabradine, verapamil, lidocaine, | | |
| | and procainamide | | |
| P3 | Amiodarone, metoprolol, | Immature | Only after |
| | ivabradine, verapamil, lidocaine, | | removal of |
| | and procainamide with removal of | | ivabradine |
| | ivabradine on day 7 post- | | |
| | transplantation | | |
| P4 | Amiodarone, metoprolol, ivabradine, | Immature | Yes; less |
| | verapamil, and lidocaine | | severe |
| | | | than P1 |
| P5 | Amiodarone, metoprolol, ivabradine, | Immature | Yes; less |
| | verapamil, and procainamide | | severe |
| | | | than P1 |
| P6 | Amiodarone, metoprolol, ivabradine, | Mature | No |
| | and mexiletine | | |

### Example 2. Antiarrhythmic Drug Combinations for Preventing Graft-Induced Arrhythmia

Pluripotent stem cell-derived cardiomyocytes (PSC-CMs) have been used in small and large animal models for their potential to regenerate injured hearts. The PSC-CMs can engraft and survive within the scar region of various animal models and can improve fractional shortening percentage (FS %)>5% in small animal models compared to vehicle alone. However, graft-related arrhythmias are observed following transplantation of PSC-CMs in large animal models, such as non-human primates and swine. In swine, the graft-related arrhythmias are more frequent and lethal compared to those in small animal models or those with higher baseline heartrates (such as macaques). Currently, significantly reducing/eliminating graft-related arrhythmias remains a top priority to using PSC-CMs as a viable therapy to treat patients in heart failure. A combinational drug therapy approach and/or maturing the PSC-CMs in vitro can be a solution to this problem. The list of all drugs in varying combinations are:

TABLE 2-1
list of drugs

| | |
|---|---|
| Amiodarone PO 200 mg | DIN 02243836 |
| Amiodarone IV 50 mg/mL | DIN 02245248 |
| Metoprolol 50 mg | DIN 00618632 |
| Ivabradine 5 mg | DIN 02459973 |
| Mexiletine 200 mg | DIN 02230360 |
| Lidocaine 2% | DIN 02422026 |
| Procainamide 100 mg/mL | DIN 02184486 |
| Verapamil 120 mg | DIN 02166739 |

In addition, both immature and in vitro matured human induced PSC-CMs (BR-immature CMs and BR-mature CMs respectively) may be used. Risk of graft-related arrhythmias is most severe when using day 14 immature PSC-CMs.

BR-matured cardiomyocytes would be delivered directly into the scar region through transepicardial injections in swine 5-weeks post myocardial infarction (MI). Severe graft-related arrhythmias (heart rate >250 bpm) would be observed following delivery of BR-immature CMs without any treatment with any antiarrhythmic drugs. The severity of graft-related arrhythmias would be reduced when using BR-matured CMs compared to day 14 immature CMs. In order to ensure proper trough levels, amiodarone levels would be measured, and dose adjusted to maintain blood levels between 1.0-2.0 mg/L. Below are examples of various combinations of antiarrhythmic drug treatment plans in swine.

Significant non-sustained and sustained VT would be observed following delivery of matured BR-CMs in the presence of antiarrhythmic drug combinations outlined in Table 5, Table 6, or Table 7. In contrast, significant non-sustained or sustained VTs would not be observed in animals treated with antiarrhythmic drug combinations outlined Tables 1 through 4.
1. No significant sustained VT would be observed when pigs would be treated with the full panel of antiarrhythmic drug treatment plan (outlined in Table 1) following delivery of immature or matured BR-CMs.

TABLE 1
Full list of antiarrhythmic drug treatment plan.

| | Pre-treatment | | |
|---|---|---|---|
| | (4-wks prior to | 1 day prior to | Day of cell |
| Drug name | cell therapy) | cell therapy | delivery |
| Amiodarone | 600 mg BID | 800 mg BID | 1-3 g BID |
| Metoprolol | | 25 mg SR BID | 50 mg SR BID |
| Ivabradine | | 5 mg BID | 10-15 mg BID |
| Lidocaine | | | 0.75-1 mg/min IV |
| Procainamide | | | 0.75-1 mg/min IV |
| Verapamil | | | 120 mg SID |

2. No significant sustained VT would be observed when pigs would be treated with a reduced cocktail plan (Table 2) following delivery of matured BR-CMs. In this example, lidocaine (IV) would be substituted for mexiletine (PO) and verapamil would be removed from the regimen.

TABLE 2
Antiarrhythmic 5-drug cocktail plan

| | Pre-treatment | | |
|---|---|---|---|
| | (4-wks prior to | 1 day prior to | Day of cell |
| Drug name | cell therapy) | cell therapy | delivery |
| Amiodarone | 600 mg BID | 800 mg BID | 1-3 g BID |
| Metoprolol | | 25 mg SR BID | 50 mg SR BID |
| Ivabradine | | 5 mg BID | 10-15 mg BID |
| Mexiletine | | | 200 mg BID |
| Procainamide | | | 0.75-1 mg/min IV |

3. No significant sustained VT would be observed when pigs would be treated with a further reduced cocktail plan (Table 3) following delivery of matured BR-CMs. In this example, procainamide would be removed compared to table 2.

TABLE 3
Antiarrhythmic 4-drug cocktail plan

| | Pre-treatment | | |
|---|---|---|---|
| | (4-wks prior to | 1 day prior to | Day of cell |
| Drug name | cell therapy) | cell therapy | delivery |
| Amiodarone | 600 mg BID | 800 mg BID | 1-3 g BID |
| Metoprolol | | 25 mg SR BID | 50 mg SR BID |
| Ivabradine | | 5 mg BID | 10-15 mg BID |
| Mexiletine | | NA | 200 mg BID |

4. No significant sustained VT would be observed following delivery of matured BR-CMs using the 3-drug treatment plan outlined in Table 4. In this example, procainamide would be removed compared to table 2.

TABLE 4
Antiarrhythmic 3-drug cocktail plan

| | Pre-treatment | | |
|---|---|---|---|
| | (4-wks prior to | 1 day prior to | Day of cell |
| Drug name | cell therapy) | cell therapy | delivery |
| Amiodarone | 600 mg BID | 800 mg BID | 1-3 g BID |
| Metoprolol | | 25 mg SR BID | 50 mg SR BID |
| Ivabradine | | 5 mg BID | 10-15 mg BID |

5. Significant non-sustained and sustained VT would be observed following delivery of matured BR-CMs using the treatment plan outlined in Table 5, Table 6, or Table 7, illustrating the importance of ivabradine (Table 5), amiodarone (Table 6), and metoprolol (Table 7) in reducing graft-related arrythmias.

TABLE 5
Antiarrhythmic treatment plan with amiodarone and metoprolol

| | Pre-treatment | | |
|---|---|---|---|
| | (4-wks prior to | 1 day prior to | Day of cell |
| Drug name | cell therapy) | cell therapy | delivery |
| Amiodarone | 600 mg BID | 800 mg BID | 1-3 g BID |
| Metoprolol | | 25 mg SR BID | 50 mg SR BID |

TABLE 6
Antiarrhythmic treatment plan with ivabradine and metoprolol

| | | | |
|---|---|---|---|
| | Pre-treatment | | |
| | (4-wks prior to | 1 day prior to | Day of cell |
| Drug name | cell therapy) | cell therapy | delivery |

| | | |
|---|---|---|
| Metoprolol | 25 mg SR BID | 50 mg SR BID |
| Ivabradine | 5 mg BID | 10-15 mg BID |

TABLE 7
Antiarrhythmic treatment plan with amiodarone and ivabradine

| | Pre-treatment | | |
|---|---|---|---|
| | (4-wks prior to | 1 day prior to | Day of cell |
| Drug name | cell therapy) | cell therapy | delivery |
| Amiodarone | 600 mg BID | 800 mg BID | 1-3 g BID |
| Ivabradine | | 5 mg BID | 10-15 mg BID |

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects described herein, is/are referred to as comprising particular elements and/or features, some embodiments described herein or aspects described herein consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments described herein, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment described herein can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A method comprising administering two or more of:
amiodarone,
metoprolol,
ivabradine, and
mexiletine,
optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof,
to a subject in need thereof, wherein the subject is in need of or has received a cardiac graft.

2. The method of claim 1, wherein the method comprises administering two or more of:
amiodarone,
metoprolol, and
ivabradine,
optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof,
to a subject in need thereof, wherein the subject is in need of or has received a cardiac graft.

3. The method of claim 1 or 2, wherein the method comprises administering
amiodarone, and
ivabradine,
optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof.

4. The method of any one of claims 1-3, wherein the method does not comprise administering amiodarone, or an analog or derivative thereof, or a pharmaceutically acceptable salt, isotopically labeled compound, tautomer, stereoisomer, solvate, tautomer, hydrate, polymorph, co-crystal, or prodrug thereof, to the subject in need thereof.

5. The method of any one of claims 1-4, wherein the method does not comprise administering ivabradine, or an analog or derivative thereof, or a pharmaceutically acceptable salt, isotopically labeled compound, tautomer, stereoisomer, solvate, tautomer, hydrate, polymorph, co-crystal, or prodrug thereof, to the subject in need thereof.

6. The method of any one of claims 1-3, comprising administering each of
amiodarone,
metoprolol,
verapamil, and
ivabradine, and
optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof,
to the subject in need thereof.

7. The method of any one of claim 1-3, or 6, further comprising administering lidocaine, or an analog or derivative thereof, or a pharmaceutically acceptable salt, isotopically labeled compound, tautomer, stereoisomer, solvate, tautomer, hydrate, polymorph, co-crystal, or prodrug thereof, to the subject in need thereof.

8. The method of any one of claim 1-3, or 6-7, further comprising administering procainamide, or an analog or derivative thereof, or a pharmaceutically acceptable salt, isotopically labeled compound, tautomer, stereoisomer, solvate, tautomer, hydrate, polymorph, co-crystal, or prodrug thereof, to the subject in need thereof.

9. The method of any one of claim 1-3, or 6-8, further comprising administering verapamil, or an analog or derivative thereof, or a pharmaceutically acceptable salt, isotopically labeled compound, tautomer, stereoisomer, solvate, tautomer, hydrate, polymorph, co-crystal, or prodrug thereof, to the subject in need thereof.

10. The method of any one of claim 1-3, or 6-9, comprising administering each of
amiodarone,
metoprolol,
verapamil,
ivabradine, and
procainamide,
optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof,
to the subject in need thereof.

11. The method of claim 10, wherein each of amiodarone, metoprolol, verapamil, ivabradine, lidocaine, and procainamide, optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof, are administered at least once per day to the subject in need thereof.

12. The method of any one of claims 1-3, comprising administering each of amiodarone, metoprolol, ivabradine, and mexiletine, optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof, to the subject in need thereof.

13. The method of claim 12, wherein each of each of amiodarone, metoprolol, ivabradine, and mexiletine, optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof, are administered at least once per day to the subject in need thereof.

14. The method of any one of claims 1-13, comprising: administering two or more of
metoprolol,
ivabradine, and/or
mexiletine,
optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof,
to the subject in need thereof.

15. The method of any one of claims 1-14, comprising administering two or more of
amiodarone,
metoprolol, and/or
mexiletine,
optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof,
to the subject in need thereof.

16. The method of any one of claims 12-15, comprising administering each of
amiodarone,
mexiletine,
verapamil,
ivabradine,
lidocaine, and
procainamide,
optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof,
to the subject in need thereof.

17. The method of any one of claims 1-16, wherein the subject is at risk of or suffers from arrhythmia.

18. The method of claim 17, wherein the arrhythmia is tachycardia.

19. The method of claim 17 or 18, wherein the arrhythmia is ventricular tachycardia.

20. The method of any one of claims 17-19, wherein the arrhythmia is caused by the cardiac graft or at least in part by the cardiac graft.

21. The method of any one of claims 17-20, wherein the arrhythmia occurs after the cardiac graft.

22. The method of any of claims 1-21, wherein the
amiodarone,
metoprolol,
ivabradine,
mexiletine,
lidocaine,
verapamil, and/or
procainamide,
optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof,
are administered concurrent with administration of the cardiac graft.

23. The method of any of claims 1-22, wherein the one or more of
amiodarone,
metoprolol,
ivabradine,
mexiletine,
lidocaine,
verapamil, and/or
procainamide,
optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof,
are administered subsequent to administration of the cardiac graft.

24. The method of any of claims 12-23, wherein the
amiodarone,
metoprolol,
ivabradine,
mexiletine,
lidocaine,
verapamil, and/or
procainamide,
optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof,
are administered prior to administration of the cardiac graft.

25. The method of any one of claims 12-24, wherein the
amiodarone,
metoprolol,
ivabradine,
mexiletine,
lidocaine,
verapamil, and/or
procainamide,
optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof,
are administered within about 7 days, or about 6 days, or about 5 days, or about 4 days, or about 3 days, or about 2 days, or about 1 days, or about 24 hours, or about 20 hours, or about 18 hours, or about 16 hours, or about 14 hours, or about 12 hours, or about 10 hours, or about 8 hours, or about 6 hours, or about 5 hours, or about 4 hours, or about 3 hours, or about 2 hours, or about 1 hour, or about 50 minutes, or about 40 minutes, or about 30 minutes, or about 20 minutes, or about 10 minutes, or about 5 minutes, or about 4 minutes, or about 3 minutes, or about 2 minutes, or about 1 minute of administration of the cardiac graft.

26. The method of any one of claims 12-24, wherein the
amiodarone,
metoprolol,
ivabradine,
mexiletine,
lidocaine,
verapamil, and/or
procainamide,
optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof,
are administered within between 1 and 3 minutes, between 3 and 10 minutes, between 10 and 30 minutes, between 30 and 60 minutes, between 1 and 3 hours, between 3 and 6 hours, between 6 and 8 hours, between 8 and 12 hours, between 12 and 18 hours, between 18 and 24 hours, between 1 and 2 days, between 2 and 3 days, between 3 and 5 days, or between 5 and 7 days, inclusive.

27. The method of any one of claims 1-26, wherein the method further comprises an additional administration of mexiletine, or an analog or derivative thereof, or a pharmaceutically acceptable salt, isotopically labeled compound, tautomer, stereoisomer, solvate, tautomer, hydrate, polymorph, co-crystal, or prodrug thereof, conducted 24 or more hours subsequent to a first administration of mexiletine, or an analog or derivative thereof, or a pharmaceutically acceptable salt, isotopically labeled compound, tautomer, stereoisomer, solvate, tautomer, hydrate, polymorph, co-crystal, or prodrug thereof.

28. The method of any one of claims 1-27, wherein the amiodarone, or an analog or derivative thereof, or a pharmaceutically acceptable salt, isotopically labeled compound, tautomer, stereoisomer, solvate, tautomer, hydrate, polymorph, co-crystal, or prodrug thereof is administered parenterally.

29. The method of any one of claims 1-28, wherein the amiodarone, or an analog or derivative thereof, or a pharmaceutically acceptable salt, isotopically labeled compound, tautomer, stereoisomer, solvate, tautomer, hydrate, polymorph, co-crystal, or prodrug thereof, is administered orally.

30. The method of any one of claims 1-29, wherein the metoprolol, or an analog or derivative thereof, or a pharmaceutically acceptable salt, isotopically labeled compound, tautomer, stereoisomer, solvate, tautomer, hydrate, polymorph, co-crystal, or prodrug thereof, is administered parenterally.

31. The method of any one of claims 1-30, wherein the metoprolol, or an analog or derivative thereof, or a pharmaceutically acceptable salt, isotopically labeled compound, tautomer, stereoisomer, solvate, tautomer, hydrate, polymorph, co-crystal, or prodrug thereof, is administered orally.

32. The method of any one of claims 1-31, wherein the metoprolol, or an analog or derivative thereof, or a pharmaceutically acceptable salt, isotopically labeled compound, tautomer, stereoisomer, solvate, tautomer, hydrate, polymorph, co-crystal, or prodrug thereof, is in an extended-release form.

33. The method of any one of claims 1-32, wherein the ivabradine, or an analog or derivative thereof, or a pharmaceutically acceptable salt, isotopically labeled compound, tautomer, stereoisomer, solvate, tautomer, hydrate, polymorph, co-crystal, or prodrug thereof, is administered parenterally.

34. The method of any one of claims 1-33, wherein the ivabradine, or an analog or derivative thereof, or a pharmaceutically acceptable salt, isotopically labeled compound, tautomer, stereoisomer, solvate, tautomer, hydrate, polymorph, co-crystal, or prodrug thereof, is administered orally.

35. The method of any one of claims 1-34, wherein the mexiletine, or an analog or derivative thereof, or a pharmaceutically acceptable salt, isotopically labeled compound, tautomer, stereoisomer, solvate, tautomer, hydrate, polymorph, co-crystal, or prodrug thereof, is administered parenterally.

36. The method of any one of claims 1-35, wherein the mexiletine, or an analog or derivative thereof, or a pharmaceutically acceptable salt, isotopically labeled compound, tautomer, stereoisomer, solvate, tautomer, hydrate, polymorph, co-crystal, or prodrug thereof, is administered orally.

37. The method of any one of claims 1-36, wherein the lidocaine, or an analog or derivative thereof, or a pharmaceutically acceptable salt, isotopically labeled compound, tautomer, stereoisomer, solvate, tautomer, hydrate, polymorph, co-crystal, or prodrug thereof, is administered parenterally.

38. The method of any one of claims 1-37, wherein the lidocaine, or an analog or derivative thereof, or a pharmaceutically acceptable salt, isotopically labeled compound, tautomer, stereoisomer, solvate, tautomer, hydrate, polymorph, co-crystal, or prodrug thereof, is administered intravenously.

39. The method of any one of claims 1-38, wherein the lidocaine, or an analog or derivative thereof, or a pharmaceutically acceptable salt, isotopically labeled compound, tautomer, stereoisomer, solvate, tautomer, hydrate, polymorph, co-crystal, or prodrug thereof, is administered orally.

40. The method of any one of claims 1-39, wherein the procainamide, or an analog or derivative thereof, or a pharmaceutically acceptable salt, isotopically labeled compound, tautomer, stereoisomer, solvate, tautomer, hydrate, polymorph, co-crystal, or prodrug thereof, is administered parenterally.

41. The method of any one of claims 1-40, wherein the procainamide, or a pharmaceutically acceptable salt, isotopically labeled compound, tautomer, stereoisomer, solvate, tautomer, hydrate, polymorph, co-crystal, or prodrug thereof, is administered orally.

42. The method of any one of claims 1-41, wherein the procainamide, or a pharmaceutically acceptable salt, isotopically labeled compound, tautomer, stereoisomer, solvate, tautomer, hydrate, polymorph, co-crystal, or prodrug thereof, is administered parenterally.

43. The method of any one of claims 1-42, wherein the procainamide, or a pharmaceutically acceptable salt, isotopically labeled compound, tautomer, stereoisomer, solvate, tautomer, hydrate, polymorph, co-crystal, or prodrug thereof, is administered orally.

44. The method of any one of claims 28-43, wherein the parenteral administration is intravenous administration.

45. The method of any one of claims 1-44, wherein the dose of amiodarone, or an analog or derivative thereof, or a pharmaceutically acceptable salt, isotopically labeled compound, tautomer, stereoisomer, solvate, tautomer, hydrate, polymorph, co-crystal, or prodrug thereof, is 100-1400 mg per day.

46. The method of any one of claims 1-44, wherein the dose of amiodarone, or an analog or derivative thereof, or a pharmaceutically acceptable salt, isotopically labeled compound, tautomer, stereoisomer, solvate, tautomer, hydrate, polymorph, co-crystal, or prodrug thereof, is about 800 mg per day.

47. The method of any one of claims 1-44, wherein the dose of amiodarone, or an analog or derivative thereof, or a pharmaceutically acceptable salt, isotopically labeled compound, tautomer, stereoisomer, solvate, tautomer, hydrate, polymorph, co-crystal, or prodrug thereof, is about 1200 mg per day.

48. The method of any one of claims 1-47, wherein the dose of metoprolol, or an analog or derivative thereof, or a pharmaceutically acceptable salt, isotopically labeled compound, tautomer, stereoisomer, solvate, tautomer, hydrate, polymorph, co-crystal, or prodrug thereof, is 75-125 mg per day.

49. The method of any one of claims 1-47, wherein the dose of metoprolol, or an analog or derivative thereof, or a pharmaceutically acceptable salt, isotopically labeled compound, tautomer, stereoisomer, solvate, tautomer, hydrate, polymorph, co-crystal, or prodrug thereof, is about 100 mg per day.

50. The method of any one of claims 1-49, wherein the dose of ivabradine, or an analog or derivative thereof, or a pharmaceutically acceptable salt, isotopically labeled compound, tautomer, stereoisomer, solvate, tautomer, hydrate, polymorph, co-crystal, or prodrug thereof, is 5-20 mg per day.

51. The method of any one of claims 1-49, wherein the dose of ivabradine, or an analog or derivative thereof, or a pharmaceutically acceptable salt, isotopically labeled compound, tautomer, stereoisomer, solvate, tautomer, hydrate, polymorph, co-crystal, or prodrug thereof, is about 10 mg per day.

52. The method of any one of claims 1-51, wherein the dose of mexiletine, or an analog or derivative thereof, or a pharmaceutically acceptable salt, isotopically labeled compound, tautomer, stereoisomer, solvate, tautomer, hydrate, polymorph, co-crystal, or prodrug thereof, is 100-500 mg per day.

53. The method of any one of claims 1-51, wherein the dose of mexiletine, or an analog or derivative thereof, or a pharmaceutically acceptable salt, isotopically labeled compound, tautomer, stereoisomer, solvate, tautomer, hydrate, polymorph, co-crystal, or prodrug thereof, is about 200-400 mg per day.

54. The method of any one of claims 1-51, wherein the dose of mexiletine, or an analog or derivative thereof, or a pharmaceutically acceptable salt, isotopically labeled compound, tautomer, stereoisomer, solvate, tautomer, hydrate, polymorph, co-crystal, or prodrug thereof, is about 200 mg per day.

55. The method of any one of claims 1-51, wherein the dose of mexiletine, or an analog or derivative thereof, or a pharmaceutically acceptable salt, isotopically labeled compound, tautomer, stereoisomer, solvate, tautomer, hydrate, polymorph, co-crystal, or prodrug thereof, is about 400 mg per day.

56. The method of any one of claims 1-55, wherein the dose of verapamil, or an analog or derivative thereof, or a pharmaceutically acceptable salt, isotopically labeled compound, tautomer, stereoisomer, solvate, tautomer, hydrate, polymorph, co-crystal, or prodrug thereof, is 150-500 mg per day.

57. The method of any one of claims 1-55, wherein the dose of verapamil, or an analog or derivative thereof, or a pharmaceutically acceptable salt, isotopically labeled compound, tautomer, stereoisomer, solvate, tautomer, hydrate, polymorph, co-crystal, or prodrug thereof, is about 240-360 mg per day.

58. The method of any one of claims 1-57, wherein the dose of amiodarone, or an analog or derivative thereof, or a pharmaceutically acceptable salt, isotopically labeled compound, tautomer, stereoisomer, solvate, tautomer, hydrate, polymorph, co-crystal, or prodrug thereof, administered is about 400 mg twice a day.

59. The method of any one of claims 1-57, wherein the dose of amiodarone, or an analog or derivative thereof, or a pharmaceutically acceptable salt, isotopically labeled compound, tautomer, stereoisomer, solvate, tautomer, hydrate, polymorph, co-crystal, or prodrug thereof, administered is about 600 mg twice a day.

60. The method of any one of claims 1-59, wherein the dose of metoprolol, or an analog or derivative thereof, or a pharmaceutically acceptable salt, isotopically labeled compound, tautomer, stereoisomer, solvate, tautomer, hydrate, polymorph, co-crystal, or prodrug thereof, administered is about 50 mg twice a day.

61. The method of any one of claims 1-60, wherein the dose of ivabradine, or an analog or derivative thereof, or a pharmaceutically acceptable salt, isotopically labeled compound, tautomer, stereoisomer, solvate, tautomer, hydrate, polymorph, co-crystal, or prodrug thereof, administered is about 5 mg twice a day.

62. The method of any one of claims 1-61, wherein the dose of mexiletine, or an analog or derivative thereof, or a pharmaceutically acceptable salt, isotopically labeled compound, tautomer, stereoisomer, solvate, tautomer, hydrate, polymorph, co-crystal, or prodrug thereof, administered is about 100-200 mg twice a day.

63. The method of any one of claims 1-62, wherein the dose of verapamil, or an analog or derivative thereof, or a pharmaceutically acceptable salt, isotopically labeled compound, tautomer, stereoisomer, solvate, tautomer, hydrate, polymorph, co-crystal, or prodrug thereof, administered is about 80-120 mg three times a day.

64. The method of any one of claim 1-63, wherein the
amiodarone,
metoprolol,
ivabradine,
mexiletine,
lidocaine,
verapamil, and/or
procainamide,
optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof,
are administered to the subject to maintain a resting heart rate of less than or equal to 150 beats per minute.

65. The method of any one of claims 1-64, wherein the
amiodarone,
metoprolol,
ivabradine,
mexiletine,
lidocaine,
verapamil, and/or
procainamide,
optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof,
are administered to the subject at least once per day until the arrhythmia has ceased.

66. The method of any one of claims 1-65, wherein the
amiodarone,
metoprolol,
ivabradine,
mexiletine,
lidocaine,
verapamil, and/or
procainamide,
optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof,
are administered to the subject for about 1-12 weeks, or about 1-8 weeks, or about 1-4 weeks after the cardiac graft is administered.

67. The method of any one of claims 1-66, wherein the cardiac graft comprises cardiomyocytes.

68. The method of claim 67, wherein the cardiomyocytes are ventricular cardiomyocytes.

69. The method of claim 68, wherein the ventricular cardiomyocytes are immature cardiomyocytes.

70. The method of claim 69, wherein the ventricular cardiomyocytes are mature cardiomyocytes.

71. The method of any one of claims 67-70, wherein the cardiomyocytes are made from induced pluripotent stem cells.

72. The method of claim 71, comprising administering the cardiomyocytes to the subject in an amount effective to treat a cardiovascular disease, disorder, or symptom thereof.

73. The method of claim 72, wherein the effective amount of cardiomyocytes is an amount effective to treat heart failure.

74. The method of any one of claims 1-73, wherein the method further comprises administering to the subject in need thereof one or more immunosuppressants.

75. The method of claim 74, wherein at least one of the immunosuppressants is cyclosporin A, methylprednisolone, or abatacept, or a pharmaceutically acceptable salt, isotopically labeled compound, tautomer, stereoisomer, solvate, tautomer, hydrate, polymorph, co-crystal, or prodrug thereof.

76. The method of claim 74 or 75, wherein at least one of the immunosuppressants is administered prior to, e.g., between 1 and 3 hours, between 3 and 6 hours, between 6 and 12 hours, between 12 and 24 hours, between 1 and 2 days, between 2 and 3 days, between 3 and 5 days, between 5 and 7 days, between 7 and 10 days, or between 10 and 14 days, inclusive, prior to the administration of the cardiac graft.

77. The method of claim 74 or 75, wherein at least one of the immunosuppressants is administered subsequent to, e.g., between 1 and 3 hours, between 3 and 6 hours, between 6 and 12 hours, between 12 and 24 hours, between 1 and 2 days, between 2 and 4 days, between 4 and 7 days, between 1 and 2 weeks, between 2 and 4 weeks, or between 1 and 2 months, inclusive, subsequent to the administration of the cardiac graft.

78. The method of any one of claims 1-77 further comprising using or implanting a mechanical circulatory support on or to the subject.

79. The method of claim 78, wherein the mechanical circulatory support is a left ventricular assist device, a right ventricular assist device, a bi ventricular assist device, extra corporeal membrane oxygenation, or implantable cardiac defibrillator, or a combination thereof.

80. The method of any one of claims 1-79, wherein the subject is at risk of or suffers from a cardiovascular disease.

81. The method of claim 80, wherein the cardiovascular disease is ischemic cardiomyopathy, dilated cardiomyopathy, or heart failure associated with prior myocardial infarction.

82. The method of any one of claims 1-81, wherein the subject is at risk of or has suffered from myocardial infarction.

83. The method of any one of claims 1-82, wherein the subject suffers from a cardiac birth defect or congenital heart disease, optionally hypoplastic left heart syndrome.

84. A pharmaceutical composition comprising two or more of:
amiodarone,
metoprolol,
ivabradine,
mexiletine,
lidocaine,
verapamil, and
procainamide,
optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof; and
optionally a pharmaceutically acceptable excipient.

85. A kit comprising:
(1) two or more of:
amiodarone,
metoprolol,
ivabradine, and/or
mexiletine,
optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof; and
(2) instructions for administering (1) to treat or prevent arrhythmia in a subject in need thereof.

86. The kit of claim 85, further comprising:
(3) lidocaine,
verapamil, and/or
procainamide,
optionally each of which independently comprises an analog or derivative thereof, or a pharmaceutically acceptable salt, isotopically labeled compound, tautomer, stereoisomer, solvate, tautomer, hydrate, polymorph, co-crystal, or prodrug thereof; and
(4) instructions for administering (3) to treat or prevent arrhythmia in a subject in need thereof.

87. The kit of claim 85 or 86, wherein the subject in need thereof is in need of or has received a cardiac graft.
